(19) United States
(12) Patent Application Publication
(10) Pub. No.: US 2018/0000778 A1
(43) Pub. Date: Jan. 4, 2018

(54) SMALL MOLECULE ANTAGONISTS OF DUSP5 AND METHODS OF USE

(71) Applicants: THE MEDICAL COLLEGE OF WISCONSIN, INC., Milwaukee, WI (US); MARQUETTE UNIVERSITY, Milwaukee, WI (US); CONCORDIA UNIVERSITY, INC., Mequon, WI (US)

(72) Inventors: Ramani Ramchandran, Pewaukee, WI (US); Daniel Sem, New Berlin, WI (US); Rajendra Rathore, Wauwatosa, WI (US)

(21) Appl. No.: 15/542,364

(22) PCT Filed: Jan. 15, 2016
(86) PCT No.: PCT/US2016/013593

§ 371 Date: Jul. 7, 2017

Related U.S. Application Data
(60) Provisional application No. 62/103,702, filed on Jan. 15, 2015.

(51) Int. Cl. A61K 31/404; A61K 31/185; C12Q 1/42

(52) U.S. Cl. CPC A61K 31/404; C12Q 1/42; C12Y 301/03016; A61K 31/185; G01N 2500/04; G01N 2500/20; G01N 2333/916

(57) ABSTRACT

The present invention is directed to compounds that specifically target DUSP5 and act as antagonists of that enzyme. Such compounds are useful in the treatment of various conditions including, but not limited to vascular anomalies, cancer, and macular degeneration.

### CROSS-REFERENCE TO RELATED APPLICATIONS

This application claims the benefit of U.S. Provisional Patent Application No. 62/103,702, filed Jan. 15, 2015, which is incorporated herein by reference as if set forth in its entirety.

### STATEMENT AS TO RIGHTS TO INVENTIONS MADE UNDER FEDERALLY SPONSORED RESEARCH AND DEVELOPMENT

This invention was made with United States government support awarded by the National Institutes of Health under Grant No. 1R01HL112639-01 and the National Heart, Lung and Blood Institute (National Institutes of Health) under Grant No. VITA/HHSN268201400009C. The government has certain rights in this invention.

### BACKGROUND

Vasculogenesis, which is the formation of new blood vessels from differentiating endothelial cells during embryonic development, is an essential feature of organ development. Inborn errors of the developmental pathways that regulate vasculogenesis result in vascular anomalies. Vascular anomalies are classified broadly into two groups, namely vascular tumors and vascular malformations. Vascular tumors, such as hemangiomas, appear after birth, grow rapidly within the first 6 months, and suddenly involute, resulting in scarring and other complications (North et al., Cardiovasc. Pathol. 15:303-317 (2006); Boon et al., Clin. Plast. Surg. 38:7-19 (2011)). Vascular malformations, on the other hand, are present at birth, are sustained throughout the life of the individual, and affect arteries, veins, and lymphatics. At present, it is often difficult to accurately diagnose and to treat these conditions.

The mitogen-activated protein kinase (MAPK) pathway is critical for cellular signaling, and proteins such as phosphatases that regulate this pathway are important for normal development of the vasculature and other tissues. Mitogen-activated protein kinases (MAPKs), such as extracellular regulated kinase (ERK), are activated by phosphorylation of tyrosine and serine/threonine residues in their activation loops. MAPKs can then be deactivated by phosphatases that remove these phosphate groups from their activation loop. Generally, protein phosphatases are enzymes that catalyze dephosphorylation of phosphoproteins and have been associated with vascular development and disease, as well as other processes such as cell cycle, DNA replication, transcription, translation, cell adhesion, glucose metabolism, smooth muscle contraction, and activation and differentiation of cells.

One such class of phosphatases, dual specific phosphatases (DUSPs), is unique in that it can dephosphorylate both serine/threonine and tyrosine residues. For example, Dual Specific Phosphatase-5 (DUSP5) plays a central role in vascular development and disease by virtue of its ability to react specifically with phosphorylated ERK (pERK). Inhibitors of various protein phosphatases could have a variety of therapeutic applications provided the inhibitor exhibits sufficient selectivity and acceptable in vivo pharmacological properties.

Despite the fact that some protein phosphatases appear to play important roles in vascular development and other phenomena related to the vasculature, very little is known about their functions. Also, many inhibitors of protein phosphatases still lack the specificity and potency desired for therapeutic use. Due to the key roles played by protein phosphatases in a number of different diseases and conditions including vascular anomalies, inhibitors of specific protein phosphatases are needed.

### SUMMARY

In a first aspect, provided herein is a pharmaceutical composition comprising a compound, or a pharmaceutically acceptable salt, solvate, or hydrate thereof, and a pharmaceutically acceptable carrier. The compound comprises two negatively charged sulfonate groups or bioisosteric sulfonate analog groups tethered by a core scaffold of biphenyl or a planar analog of biphenyl, and the core scaffold separates the negatively charged groups at a distance of about 6 to about 8 Angstroms. (Å). The core scaffold can comprise one or more of fluorene, biphenyl, carbazole, dibenzofuran, or naphthalene.

The compound can have a structure selected from the group consisting of:

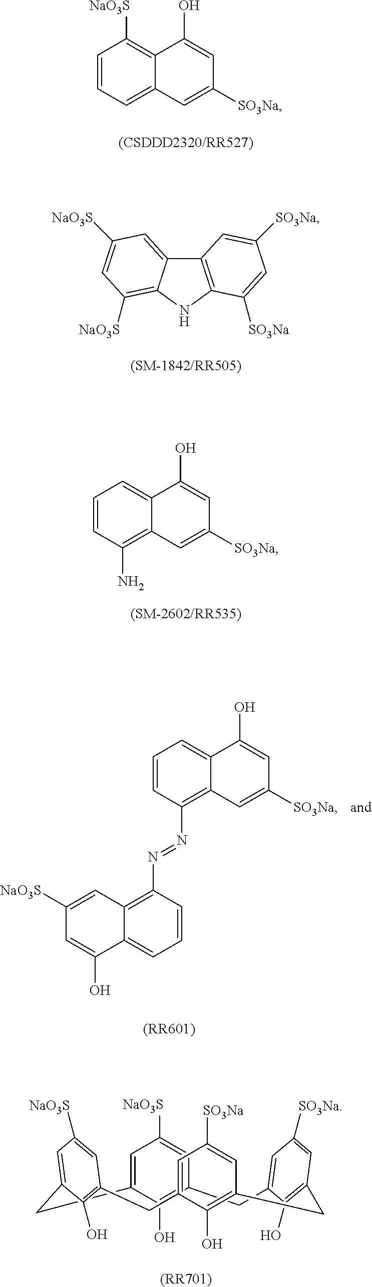

In some cases, the negatively charged sulfonate groups or bioisosteric sulfonate analog groups are separated by a distance of about 7 angstroms. The bioisosteric sulfonate analog groups can be selected from the group consisting of sulfonamide, tetrazole, and carboxylic acid. The compound can be a dual specificity phosphatase-5 (DUSP5) inhibitor.

In another aspect, provided herein is a medicament for treating, preventing, or alleviating a vascular anomaly comprising at least one compound as provided herein in an effective amount to antagonize DUSP5.

In a further aspect, provided herein is a method of treating, preventing, or alleviating a vascular anomaly comprising administering to a subject in need thereof a therapeutically effective amount of a compound or composition as provided herein, wherein the compound antagonizes dual specificity phosphatase-5 (DUSP5). The vascular anomaly can be associated with a Serine to Proline mutation at amino acid residue position 147 of DUSP5.

In another aspect, provided herein is a method of screening a compound for effectiveness as an antagonist of DUSP5. The method can comprise a) exposing a sample comprising a DUSP5 polypeptide to a compound, and b) determining if DUSP5 phosphatase activity in said sample is decreased in comparison to a control sample lacking the compound.

In a further aspect, provided herein is use of a DUSP5 inhibitor for treating, preventing, or alleviating vascular anomalies. Also provided herein is use of a DUSP5 inhibitor for the preparation of a medicament for treating, preventing, or alleviating vascular anomalies.

### INCORPORATION BY REFERENCE

All publications, patents, and patent applications mentioned in this specification are herein incorporated by reference to the same extent as if each individual publication, patent, and patent application was specifically and individually indicated to be incorporated by reference.

### BRIEF DESCRIPTION OF THE DRAWINGS

The present invention will be better understood and features, aspects and advantages other than those set forth above will become apparent when consideration is given to the following detailed description thereof.

FIGS. 1A-1B are models of DUSP5 and ERK2 domains. (A) Model depicting the two domains of DUSP5. This model is comprised of two domains, the ERK binding domain (EBD) and phosphatase domain (PD), and illustrates the relative location of the domains and their connection via a 30 amino acid linker of unknown structure. The EBD is a homology model, constructed using the solution structure (21% identity and 35% homology) of human MKP-3 protein (PDB code 1HZM) as a template (37). The PD is the previously reported crystal structure of human DUSP5 PD (PDB code 2G6Z). The 30 amino acid linker region connecting the two domains was prepared manually, and is of unknown structure. The S147P mutation present in patients with vascular anomalies is shown in green, and arginine-rich basic regions have been identified. (B) DUSP5 and ERK2 binding model. DUSP5 (green) is positioned similarly as in panel A, with the EBD to the left and PD to the right, wrapping around human ERK2. Model was prepared as described in the previous paper. See Neumann et al. (2015) BMC Biochemistry 16:19. The linker region may have the first 11 amino acids as helical based secondary structure predictions, although this was only found to be loosely helical after molecular dynamics simulations. The ERK2 structure (PDB code 3160) is shown between the DUSP5 domains to illustrate relative shape and size complementarity; and, relative orientation of ERK2 and DUSP5 is based on the molecular dynamics simulation and associated analysis presented in the previous paper.

FIGS. 2A-2D present predicted docking poses. (A) Predicted docking pose of SM1842/RR505 (gold) in DUSP5 PD (blue), using Autodock 4.2. The inset image shows predicted binding position relative to the rest of the protein. The side chains around the bound ligand (mostly arginine guanido groups) are delineated in light turquoise and the catalytic cysteine is displayed in yellow. Three-arginine residues are observed around one sulfonate group of SM1842/RR505. The predicted binding energy for this pose was −9.69 kcal/mol. (B) Optimal overlay of SM1842/RR505 (gold) and naphthalene trisulfonate (NTS, moss green), using OpenEye Scientific Software ROCs v. 3.0. (22). (C) Lowest energy binding pose for NTS (moss green) in DUSP5PD (blue), with a calculated binding energy of −8.48 kcal/mol. (D) Second lowest energy binding pose for NTS (seafoam), with a calculated binding energy of −8.21 kcal/mol.

FIG. 3 presents structures, docking energies, and IC₅₀ values of DUSP5 PD inhibitors.

FIGS. 4A-4B present Michaelis-Menten Kinetics. (A) Michaelis-Menten plot of DUSP5 PD(WT) initial velocity versus substrate (pNPP) concentration, monitoring production of para-nitrophenol at 405 nm. Reaction was in 100 mM Tris-HCl (pH 7.5), 100 mM NaCl, 5 mM MgCl2 and 1 mM DTT, and was initiated with enzyme. The line represents a nonlinear least squares fit to equation 1. (B) Enzymatic rate as a function of DMSO concentration, and at a fixed level of pNPP (5 mM), with other conditions as in panel (A). Relative enzyme activation represents the rate normalized to that obtained at 0% DMSO.

FIGS. 5A-5B present IC₅₀ measurements. (A) DUSP5 PD(WT) initial velocity versus inhibitor concentration, and fitted to equation 3 to obtain IC₅₀ values (Table 1). Conditions were as described for FIG. 3. (B) Same as panel (A), but comparing Suramin and NTS, demonstrating the affinity increase that is obtained due to tethering the NTS fragments.

FIGS. 6A-6C demonstrate the effect of detergent on Suramin inhibition. (A) NTS IC50 measurement in the presence and absence of 0.5% Triton, showing no detergent-effect on inhibition of DUSP PD(WT) pNPP phosphatase activity. (B) Suramin IC50 measurement in the presence and absence of 0.5% Triton shows a loss of some inhibitory capability in the presence of detergent. (C) Effect of increasing detergent levels (Triton X-100) on rate of DUSP5 PD(WT) in the presence of a fixed concentrations of inhibitor and substrate (at 1 μM Suramin and 5 mM pNPP). Detergent removes some, but not all, of Suramin's inhibitory effect, showing a plateau level 30% inhibition.

FIGS. 7A-7D demonstrate the effect of inhibitor on pERK levels on HUVEC cells, upon titration with: (A) Suramin, (B) SM1842, (C) RR505, and (D) and RR506. Suramin produced a significant decrease in pERK levels, while Na₃VO₄ (200 μM) produced the expected increase in pERK levels. HUVECs were incubated with the indicated drug concentrations for 2 hours and western blotted for pERK and total ERK. Sodium orthovanadate (Na₃VO₄) at 200 μM was used as a positive control. Quantification of pERK expression levels were conducted using densitometry and made relative to total ERK expression levels. Results are the mean±S.E. from three independent experiments. Statistical significance was determined by one-way ANOVA and a Dunnett's t-test. (*, p<0.05; **, p<0.01).

FIG. 8 demonstrates potential NTS binding sites on DUSP5 PD. Docking poses for 100 dockings of NTS mapped onto an electrostatic surface (blue, positive; red, negative) of DUSP5 PD. Poses were observed in three clusters, with distance from external pockets to the active site pocket shown. Clusters are observed at distances that suggest binding of Suramin such that both NTS substructures would fit into defined binding pockets on the same DUSP5 PD protein molecule seems unlikely.

FIGS. 9A-9D demonstrate pharmacophore-based identification of DUSP5 PD inhibitors. (A) Crystal structure of DUSP PD(C263 S) (12), showing the two bound sulfate ions in the two anion-binding pockets postulated to be occupied by the two phosphate groups of the ERK2 activation loop (pThr-Glu-pTyr) (12-14). The anion pocket closest to the catalytic nucleophile (Cys263) is labeled S1, and the distal anion pocket is labeled S2. The S2 anion (sulfate) is stabilized be several arginine residues, while the S1 anion may derive some helix dipole stabilization by virtue of its location at the N-terminal end of a long central helix. The sulfur to sulfur distance of 7.2 Å defines the DUSP PD pharmacophore as two anionic groups separated by ˜7 Å. Overlay of the S1-S2 pharmacophore (two sulfates, shown as purple) on RR505 (B) indicates a poor match, while overlay on NTS (C) in one of two possible orientations (related by a 1800 rotation) is better. (D) A ligand-based search using this pharmacophore identified CSD3-2320, which also matched the S1-S2 sulfate positions well. The overlay in panel (D), as in panel (C), is shown in one of the two possible orientations that optimally align active site sulfate and ligand sulfonate groups.

FIGS. 10A-10E demonstrate CSD3-2320 binding to DUSP5 PD. (A) Dose response curve for CSD3-2320 as an inhibitor of the DUSP PD phosphatase activity, using pNPP as substrate. Experimental conditions as in FIGS. 3 and 4A-4B. (B) Chemical structure of CSD3-2320. (C) DUSP5 PD (C263S) 1H-15N HSQC spectrum of DUSP5 PD (C263S) in pH 6.8, 50 mM potassium phosphate, 100 mM potassium chloride buffer. Overlay is of 500 μM 15N-labeled DUSP5 PD alone (black), and in the presence of 500 μM CSD3-2320 (red). Potentially important chemical shift perturbations due to binding are indicated using arrows. Panels (D) and (E) represent the model from FIG. 1, with CSD3-2320 positioned such that its two sulfonate groups are optimally overlaid with the two phosphate groups on the ERK2 pThr-Glu-pTyr peptide. This overlay results in the phenolic ring of the CSD3-2320 naphthalene core being superimposed directly on the tyrosine phenol ring of the pThr-Glu-pTyr peptide. In panel (E), serine-147 (which is mutated to proline in clinical biopsy isolates (2)) is visible as a stick rendering on the bottom left of the model, distal from the phosphatase domain binding site which is occupied by CSD3-2320.

FIG. 11 demonstrates IC₅₀ measurement for 2320 inhibition of full-length DUSP5. Dose response curve for 2320 as an inhibitor of the full-length DUSP phosphatase activity, using pERK2 as substrate, and quantifying ERK2 and pERK2 levels in a western blot.

FIG. 12 shows the chemical structures of exemplary DUSP5 inhibitors.

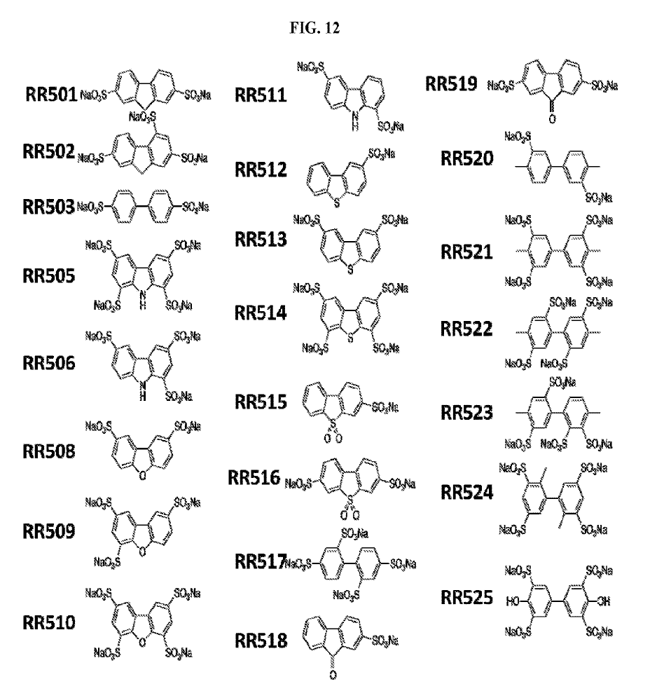

FIG. 13 shows the chemical structures of exemplary DUSP5 inhibitors.

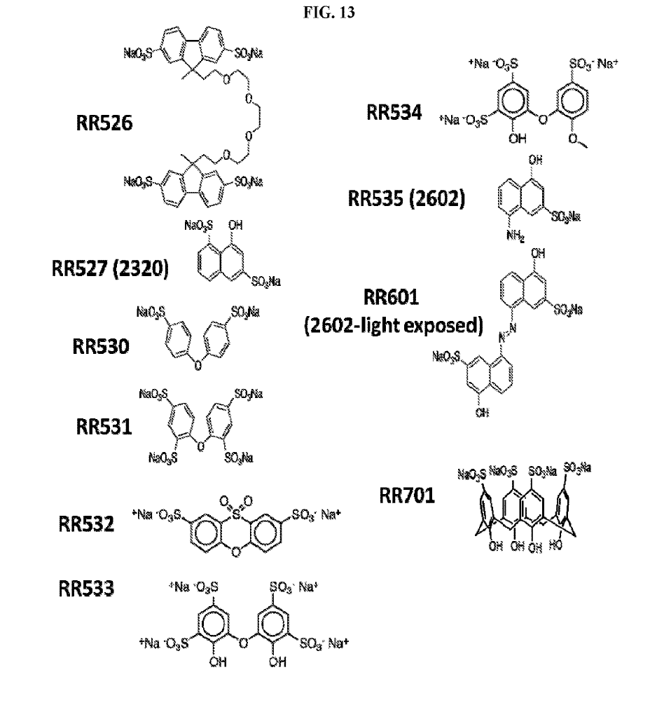

FIG. 14 shows the chemical structures of exemplary DUSP5 inhibitors.

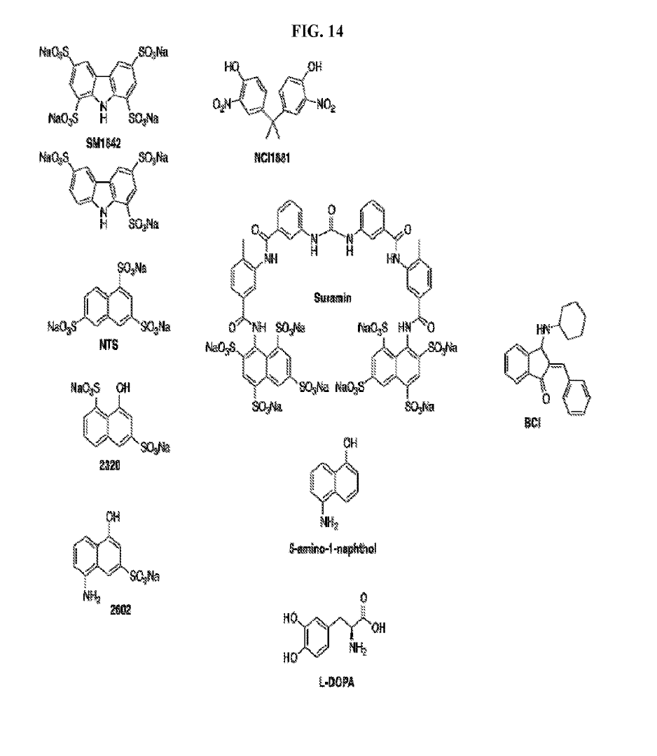

FIG. 15 presents the chemical structure and phosphatase activity data for compound RR701.

### DETAILED DESCRIPTION OF THE INVENTION

This application relates to compounds or therapeutic agents that can be used as selective inhibitors of Dual Specific Phosphatase 5 (DUSP5), such as mammalian (e.g., human) DUSP5. DUSP5 is comprised of two domains, an N-terminal ERK binding domain (EBD) and a C-terminal phosphatase domain (PD) (5, 12). While there is no structure available for intact DUSP5, a crystal structure of the PD is available. The DUSP5 PD structure has two anionic sulfate groups bound in the active site near the catalytic Cys263 (mutated to serine), and separated by 7.2 Å. These sulfates had been proposed to occupy the same binding pockets that are occupied by the phosphate groups on the substrate (12). In the case of the ERK2 substrate, this region in the DUSP5 PD would be occupied by the pThr-Glu-pTyr tripeptide region of the ERK2 activation loop (13, 14). (Nayak et al., BMC Biochem. 15:27 (2014)).

DUSP5's primary substrate is extracellular regulated kinase (pERK), and molecular dynamics simulation studies point to several key residues in the active site binding pocket, which if targeted correctly will inhibit DUSP5's ability to dephosphorylate pERK. Low molecular weight compounds (e.g., less than about 500 daltons) that selectively inhibit DUSP5 catalytic activity were identified by screening of a virtual database of compounds against the 3D model structure of DUSP5 (based on the crystal structures of the human DUSP5 phosphatase domain (PD) and ERK2) and then further screened using an in vitro phosphatase assay.

The compounds and compositions provided herein are based at least in part on the inventors discovery of a Serine to Proline mutation at amino acid residue position 147 in DUSP5 in patients afflicted with vascular defects. This mutation was found in a variety of VAs (17 out of 24 patients). The Inventors previously demonstrated that the S147P mutation interferes with the dephosphorylating activity of DUSP5 protein and makes the protein hypoactive towards pERK1/2 (Nayak et al., BMC Biochem. 15:27 (2014)). Based on the discovery of this mutation, the inventors have further identified small molecules that agonize or antagonize DUSP5 function. Molecules with both functions are beneficial for promoting or stopping proliferation of endothelial cells in disease. Designing inhibitors of DUSP5 would seem counter-intuitive at first, because blocking a signal that shuts down proliferation is likely to increase proliferation. However, in vascular tumors such as hemangiomas, a disease associated with increased endothelial cell proliferation, it has been clinically observed that increased proliferative phase leads to quicker involution phase, where the disease regresses faster.

Accordingly, in a first aspect, provided herein are compounds that antagonize DUSP5. A compound provided herein or a salt thereof has a protein phosphatase-inhibitory activity, particularly a potent DUSP5-inhibitory activity. Further, it also exerts its inhibitory activity on DUSP5 in cells (particularly, in endothelial cells). Without being bound to any particular theory, since DUSP5 is a putative suppressor of pERK signaling, modulating the DUSP5:pERK pathway using one or more compounds that antagonize DUSP5 is likely to benefit several disease conditions or symptoms thereof.

Generally, a DUSP5 antagonist provided herein comprises polysulfonated aromatic compounds and carbazole and naphthalene scaffolds. In exemplary embodiments, DUSP5 antagonist provided herein comprise two negatively charged sulfonate groups tethered by a core scaffold in such a way that a separation of ˜7 Å between them is maintained. Such a separation can be achieved by using core scaffold of biphenyl and its planar analogs (e.g., carbazole), naphthalene, and calixarene. In other cases, DUSP5 antagonists of this disclosure comprise negatively charged groups known to be bioisosteric analogs of sulfonates such as, without limitation, tetrazoles, sulfonamides, and carboxylic acids. In such cases, the DUSP5 antagonist comprises two negatively charged bioisosteric sulfonate-analog groups tethered by a core scaffold in such a way that a separation of ˜7 Å between them is maintained as described herein.

As used herein, the term “sulfonate” refers to anions with the general formula RSO₃⁻. Sulfonate are the conjugate bases of sulfonic acids having the formula RSO₂OH. The terms “bioisosteric” and “bioisostere” as used herein refer to analogs in which one or more atoms (or groups of atoms) have been substituted with replacement atoms (or groups of atoms) having similar steric and/or electronic features to those atoms which they replace. For example, the substitution of a hydrogen atom or a hydroxyl group with a fluorine atom is a commonly employed bioisosteric replacement.

The compounds provided by the present disclosure are shown, for example, above in the summary of the invention section, the preceding paragraphs, in the claims below, and in the formulas provided in the Figures. They may be made using the methods outlined in the Examples section. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein. As used herein, the term “compound” is interchangeable with the term “chemical entity.”

Preferred examples of the compound of the present invention include the DUSP5 antagonists presented in Table 1. As described in the Examples that follow, these compounds are small molecule inhibitors that occupy the active site pocket on the DUSP5 phosphatase domain (PD), and act as inhibitors of phosphatase enzymatic activity. Briefly, the inventors discovered a number of compounds that seem to affect DUSP5 levels in cells (including HUVEC cells), as well as levels of phosphorylated ERK and dephosphorylated ERK (a protein kinase). It was discovered that DUSP5 controls the on/off switch for ERK by removing a phosphate group from ERK.

TABLE 1
DUSP5 Antagonist Compounds

RR527 (CSD3-2320; also known as CSDDD2320)
SM1472, SM1693, SM2362, SM2746, SM5023
RR505 (also known as SM1842)
RR533
RR535 (also known as NCI2602)
RR601A
RR701 (calixarene)
Fluorene Derivatives: RR501, RR502
Biphenyl Derivatives: RR503
Carbazole Derivatives: RR511, RR505, RR506
Dibenzofuran Derivatives: RR507, RR508, RR509, RR510

It should be recognized that the particular anion or cation forming a part of any salt form of a DUSP5 antagonist compound provided herein is not critical, so long as the salt, as a whole, is pharmacologically acceptable. The term “pharmaceutically acceptable salt” as applied to the compounds provided herein defines any non-toxic organic or inorganic acid addition salt of the free base compound which is suitable for use in contact with mammalian tissues without undue toxicity, irritation, allergic response and which are commensurate with a reasonable benefit/risk ratio. Suitable pharmaceutically acceptable salts are well known in the art. Examples are the salts with inorganic acids (for example hydrochloric, hydrobromic, sulphuric and phosphoric acids), organic carboxylic acids (for example acetic, propionic, glycolic, lactic, pyruvic, malonic, succinic, fumaric, malic, tartaric, citric, ascorbic, maleic, hydroxymaleic, dihydroxymaleic, benzoic, phenylacetic, 4-aminobenzoic, 4-hydroxybenzoic, anthranilic, cinnamic, salicylic, 2-phenoxybenzoic, 2-acetoxybenzoic and mandelic acid) and organic sulfonic acids (for example methanesulfonic acid and p-toluenesulfonic acid). The compounds provided herein may also be converted into salts by reaction with an alkali metal halide, for example sodium chloride, sodium iodide or lithium iodide. Preferably, the compounds provided herein are converted into their salts by reaction with a stoichiometric amount of sodium chloride in the presence of a solvent such as acetone.

Additional examples of pharmaceutically acceptable salts and their methods of preparation and use are presented in Handbook of Pharmaceutical Salts: Properties, and Use (2002), which is incorporated herein by reference.

These salts and the free base compounds can exist in either a hydrated or a substantially anhydrous form. It will appreciated that many compounds can form complexes with solvents in which they are reacted or from which they are precipitated or crystallized. These complexes are known as “solvates.” Where the solvent is water, the complex is known as a “hydrate.” All solid forms of the compounds provided herein, including any solvates or hydrates thereof are within the scope of the present invention. It will also be appreciated that many compounds can exist in more than one solid form, including crystalline and amorphous forms. In general, the acid addition salts of the compounds of the invention are crystalline materials which are soluble in water and various hydrophilic organic solvents and which in comparison to their free base forms, demonstrate higher melting points and an increased solubility.

In some cases, compounds as provided herein are part of a pharmaceutically acceptable complex. As used herein, the term “pharmaceutically acceptable complex” refers to compounds or compositions in which the compound of the invention forms a component part. Thus, the complexes of the invention include derivatives in which the compound of the invention is physically associated (e.g., by covalent or non-covalent bonding) to another moiety or moieties. The term “pharmaceutically acceptable complex” therefore includes multimeric forms of the compounds of the invention. Such multimers may be generated by linking or placing multiple copies of a compound of the invention in close proximity to each other (e.g. via a scaffolding or carrier moiety).

Candidate DUSP5 antagonist compounds can be assessed for inhibitory activity using any appropriate assays. For example, a cell based assay can be used, where cells are activated with VEGF (the growth hormone that stimulate vascularization) and then contacted with a candidate compound or appropriate control. In exemplary embodiments, an in vitro phosphatase assay can be used to detect and measure the inhibitory capacity of candidate compounds. As described in the Examples below, a p-nitrophenol phosphate (pNPP) based enzymatic assay can be used to screen for inhibitors of the phosphatase domain of DUSP5. Inhibitory activity can be determined based on the “IC50,” which is inhibitory dose at which 50% of the maximum response is obtained. This quantitative measure indicates how much of a particular drug or other substance (inhibitor) is needed to inhibit a given biological, biochemical or chemical process (or component of a process, i.e. an enzyme, cell, cell, receptor) by half.

Presence of DUSP5 or pERK can be monitored in cells using fluorescently tagged antibodies against these proteins. In some cases, a cell proliferation assay can be performed to identify a candidate as a potential anti-cancer agent.

Furthermore, as the compounds provided herein are low molecular weight compounds, they are not immunogenic and can be administered orally. Thus, they are useful as medicine for mammals, preferably humans (male or female), from the viewpoint of safety and compliance. Accordingly, the subject according to at least some embodiments of the present invention is a mammal, preferably a human (male or female). It is to be understood that the principles of the invention indicate that the invention is effective with respect to all mammals, which are intended to be included in the term “subject.” In this context, a mammal is understood to include any mammalian species in which treatment of diseases or conditions associated with vascular anomalies (“vascular anomaly-related disorder”) is desirable.

In a further aspect, the present invention provides therapeutic formulations comprising one or more compounds that selectively antagonize DUSP5. As described herein, diseases and conditions associated with an alterations or disruption of the DUSP5:pERK pathway can be treated with a therapeutic formulation provided herein. For example, therapeutic administration of one or more compounds that selectively antagonize DUSP5 can treat cancer-related processes such as angiogenesis, cellular proliferation, migration, invasion, and tumor survival. In addition to cancer, immune-related conditions (e.g., auto-immune disorders), metabolic disorders (e.g., diabetes), and cardiovascular diseases are conditions for which therapeutic administration of DUSP5 antagonist compounds will be beneficial.

As used herein, the terms “treat” and “treating” refer to both therapeutic treatment and prophylactic or preventative measures, wherein the object is to prevent or slow down (lessen) an undesired physiological change or disorder (e.g., arresting further development of the pathology), such as cancer progression. For purposes of this disclosure, beneficial or desired clinical results include, without limitation, an alleviation of one or more clinical indications, decreased severity of one or more clinical indications, diminishment of the extent of disease, stabilization of the disease state (i.e., not worsening, arresting further development), delay or slowing, halting, or reversing disease progression, and partial or complete remission, whether detectable or undetectable. “Treatment” also refers to prolonging survival by weeks, months, or years as compared to expected survival if not receiving treatment according to a method provided herein. Subjects in need of treatment can include those already having or diagnosed with a disease or condition as well as those prone to, likely to develop, or suspected of having the disease or condition.

In the context of cancer, the term “treating” includes any or all of: preventing growth of tumor cells, cancer cells, or of a tumor; preventing replication of tumor cells or cancer cells, lessening of overall tumor burden or decreasing the number of cancerous cells, and ameliorating one or more symptoms associated with the disease.

In the context of an autoimmune disease, the term “treating” includes any or all of: preventing replication of cells associated with an autoimmune disease state including, but not limited to, cells that produce an autoimmune antibody, lessening the autoimmune-antibody burden and ameliorating one or more symptoms of an autoimmune disease.

As used herein, the terms “prevent” and “preventing” include (1) inhibiting the onset of a disease in a subject or patient which may be at risk and/or predisposed to the disease but does not yet experience or display any or all of the pathology or symptoms of the disease, and/or (2) slowing the onset of the pathology or symptoms of a disease in a subject or patient which may be at risk and/or predisposed to the disease but does not yet experience or display any or all of the pathology or symptoms of the disease.

Preferably, a therapeutic formulation (pharmaceutical composition) provided herein will be formulated, dosed, and administered in a fashion consistent with good medical practice. Factors for consideration in this context include the particular disorder being treated, the particular mammal (e.g., human) being treated, the clinical condition of the individual patient, the cause of the disorder, the site of delivery of the agent, the method of administration, the scheduling of administration, and other factors known to medical practitioners. Preferably, a therapeutic formulation provided herein is prepared by mixing one or more compounds that selectively antagonize DUSP5 with a physiologically acceptable carrier, excipient, or stabilizer. In exemplary embodiments, the therapeutic formulation is prepared in the form of a lyophilized formulation or aqueous solution.

In some cases, the small molecule DUSP5 antagonist provided herein may be the sole active agent in the formulation. The compound (e.g., a small molecule DUSP5 antagonist) need not be, but is optionally formulated with one or more agents currently used to prevent or treat a disease or condition associated with a vascular anomaly. For example, the therapeutic formulation may further comprise one or more other active agents or carrier moieties suitable for an intended use. For example, a pharmaceutical composition comprising a small molecule DUSP5 antagonist may also comprise one or more agents useful for treating or preventing a vascular anomaly or an anti-cancer agent.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

When including a DUSP5 antagonist compound provided herein or a salt thereof in a pharmaceutical composition, it can be mixed with pharmaceutically acceptable carriers as needed, and prepared into various dosage forms depending on the preventive or therapeutic purpose. Examples of the dosage form include an oral agent, an injection, a suppository, an ointment, and a patch, among which an oral agent is preferable. Each of these dosage forms can be produced by publicly known production methods routinely employed by those skilled in the art.

When preparing an oral solid preparation, an excipient, and if needed, a binder, a disintegrant, a lubricant, a colorant, a flavoring agent, an odor-masking agent, and the like can be added to the compound of the present invention, and then a tablet, a coated tablet, a granule, a powder, a capsule, and the like can be produced by an ordinary method.

When preparing an oral liquid preparation, a taste-masking agent, a buffer, a stabilizer, an odor-masking agent, and the like can be added to the DUSP5 antagonist compound, and then an internal liquid agent, a syrup, an elixir, and the like can be produced by an ordinary method.

When preparing an injection, a pH adjuster, a buffer, a stabilizing agent, an isotonic agent, a local anesthetic, and the like can be added to the compound of the present invention, and then injections for subcutaneous, intramuscular, and intravenous administration can be produced by an ordinary method.

A suppository can be prepared by adding a pharmaceutical carrier publicly known in the art, for example, polyethylene glycol, lanolin, cacao butter, and fatty acid triglyceride to the DUSP5 antagonist compound, and then applying an ordinary production method.

When preparing an ointment, a normally-used base, stabilizer, humectant, preservative, and the like are added to the compound of the present invention as needed. The resulting mixture is then mixed and prepared into an ointment by an ordinary method.

When preparing a patch, the aforementioned ointment, a cream, a gel, a paste, and the like may be spread over a normal base by an ordinary method.

The content of the compound of the present invention in each of the aforementioned preparations varies depending on the symptoms of a patient, the dosage form, and the like; however, normally, the content in an oral agent is approximately 0.05 to 1000 mg, the content in an injection is approximately 0.01 to 500 mg, and the content in a suppository is approximately 1 to 1000 mg.

Also, the daily doses of these preparations vary depending on the symptoms, body weight, age, sex, and the like of a patient, and thus cannot be flatly determined; however, normally, the daily dose of an adult (a body weight of 60 kg) is approximately 0.05 to 5000 mg, preferably 0.1 to 1000 mg, and the daily dose is preferably administered once a day or divided into approximately two to three portions per day.

In exemplary embodiments, the pharmaceutical composition is useful to treat a vascular anomaly-related disorder. As used herein, the term “a vascular anomaly” should be understood to encompass any birthmark and/or vascular anomaly related disorder which may appear on any part of the body both externally or in internal organs. Examples of the disease or condition associated with vascular anomalies (“vascular anomaly-related disease”) that can be treated by administration of a compounds described herein include, without limitation, vascular permeability, plasma leakage, venous malformation (VM), hemangioblastoma, hemangiomas, intramuscular hemangiomas, brain arteriovenous malformations (BAVM), arteriosclerosis, thrombosis, leukomalacia (PLV), Hereditary Hemorrhagic telangiectasia (HHT), Ataxia telangiectasia, and Osler-Weber syndrome.

In some embodiments, the subject invention further provides for use of a compound as described herein or a homolog or derivative thereof for the manufacture of a medicament. In some cases, the subject invention further provides for use of a compound for use in therapy.

As used herein, the term “about” means within 5% of a stated amount, concentration, concentration range, or stated time frame.

As used herein, the terms “antagonist” or “inhibitor” are used interchangebly and refer to a molecule which, when bound to a phosphatase such as DUSP5, blocks or modulates the biological activity of the phosphatase. Antagonists and inhibitors may include small molecules, proteins, nucleic acids, carbohydrates, or other compounds or molecules that bind to DUSP5. In some cases, an antagonist can bind a ligand but does not provoke the biological response seen when an agonist binds to the ligand. Antagonists can compete with agonists for binding to the same site on the protein ligand, or might bind to a separate site, causing the protein to undergo changes that preclude agonist binding. Antagonists can be used therapeutically to block an overactive agonist/ligand interaction that is causing an undesired biological consequence.

As used herein, the term “modulate” refers to a change or an alteration in the biological activity of a phosphatase such as DUSP5. Modulation may be an increase or a decrease in protein activity, a change in binding characteristics, or any other change in the biological, functional, or immunological properties of the phosphatase.

It must be noted that as used herein and in the appended claims, the singular forms “a,” “an,” and “the” include plural reference unless the context clearly dictates otherwise. As well, the terms “a” (or “an”), “one or more” and “at least one” can be used interchangeably herein. It is also to be noted that the terms “comprising,” “including,” and “having” can be used interchangeably.

In the specification and in the claims, the terms “including” and “comprising” are open-ended terms and should be interpreted to mean “including, but not limited to . . . .” These terms encompass the more restrictive terms “consisting essentially of” and “consisting of.”

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications and patents specifically mentioned herein are incorporated by reference for all purposes including describing and disclosing the chemicals, cell lines, vectors, animals, instruments, statistical analysis and methodologies which are reported in the publications which might be used in connection with the invention. All references cited in this specification are to be taken as indicative of the level of skill in the art.

The invention will be more fully understood upon consideration of the following non-limiting Examples, which are offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The following examples are, of course, offered for illustrative purposes only, and are not intended to limit the scope of the present invention in any way. Indeed, various modifications of the invention in addition to those shown and described herein will become apparent to those skilled in the art from the foregoing description and the following examples and fall within the scope of the appended claims.

### Example 1—Sulfonate-Based Inhibitors to Mimic DUSP5 Peptide Binding

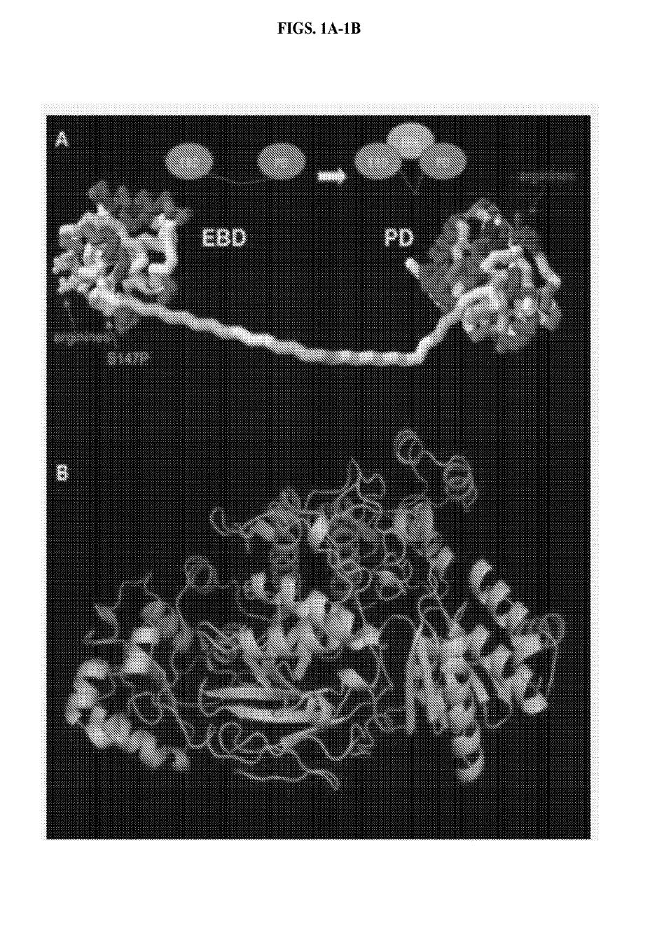

A molecular model based on the crystal structures of the human DUSP5 PD and ERK2, and a homology model of the DUSP5 EBD, illustrates the bivalent nature of the interaction between the two domains of DUSP5 and the ERK2 substrate (FIGS. 1A-1B). This model illustrates the distal location of the S147P mutation, relative to the phosphatase domain (PD). In this section, we describe identifying small molecule inhibitors that occupy the active site pocket on the DUSP5 PD, and act as inhibitors of phosphatase enzymatic activity. As described herein, we used docking screens and inhibitor verification (using a p-nitrophenol phosphate (pNPP) based enzymatic assay) to identify a series of polysulfonated aromatic inhibitors that occupy the DUSP5 active site (the phosphatase domain of DUSP5) in the region that is likely occupied by the dual-phosphorylated ERK2 substrate (pThr-Glu-pTyr). The most potent inhibitor has a naphthalene trisulfonate (NTS) core.

Ligand-based screening, based on a pharmacophore derived from the 7 Å separation of sulfonates on initial inhibitors and on sulfates present in the DUSP5 crystal structure, identified a disulfonated and phenolic naphthalene inhibitor that is more potent than NTS and does not aggregate. Specifically, we computationally docked 10,500 small molecules (SMs) to the phosphatase domain of DUSP5 and identified a small molecule designated RR505 (also known as SM1842). Modeling suggests this inhibitor may have broader relevance for mimicking the dual-phosphate loops recognized by DUSPs, as it positions sulfonates where the two phosphates on the substrate peptide (pThr-Glu-pTyr) bind, mimics the pTyr, and positions a phenolic hydroxyl where a water nucleophile would reside. As described in herein, RR505 acts at sub-micromolar range in p-ERK assay in vitro and does not appear to affect normal vasculature in a developing embryo. More significantly, RR505 appears to restore mutant DUSP5's function, which in essence makes it a useful small molecule for further drug development. Restoring mutant DUSP5 function back to wild type will be beneficial for most diseases where DUSP5-pERK axis is attenuated. This will include cancer, vascular anomalies, and immune-related conditions. If the drug only influences mutant DUSP5 activity and minimally the WT activity, this provides a unique targeting opportunity and selectivity. The drug selectivity will result in minimal off target effects for the drug.

A search for similar compounds in a drug database identified Suramin, a dimerized form of NTS. While Suramin appears to be a potent and competitive inhibitor (25+5 μM), binding more tightly than the monomeric ligands of which it is comprised, it also aggregates. This makes Suramin non-ideal as a drug lead, and raises concerns about Suramin's current clinical use. Conditions that might benefit from this small molecule include proliferative conditions associated with vasculature. A screen in a public database of commercially-available chemical compounds (the ZINC database; Irwin et al., J. Chem. Inf. Model. 52(7): 1757-68 (2012); available at zinc.docking.org on the World Wide Web) for structures similar to RR505 yielded two compounds: naphthalene 1,3,6-trisulfonate (NTS) and Suramin. Suramin has been approved for tumor clinical trial and at present, no evidence exists in the literature that suggests that DUSP5/ERK axes may be the target for this drug.

DUSP5 and pERK individually influence many signaling pathways in a cell. In general, DUSP5 blocks pERK from activating downstream signaling. DUSP5 is a putative suppressor of pERK signaling. Therefore, influencing this pathway with small molecules is likely to benefit several disease entities. Specific processes in cancer that will benefit include angiogenesis, cellular proliferation, migration, invasion, and survival. In addition metabolism, immune-related phenotypes are likely to benefit as well. Diseases ranging from auto-immune disorder to diabetes to cardiovascular diseases and cancer are possible avenues for drugs that influence the DUSP5:pERK pathway.

Molecular Docking.

The Center for Structure-based Drug Design and Development (CSD3) chemical library, consisting of 11,500 drug-like chemicals, was prepared in electronic format as two-dimensional (2D) SDF files. Using Pipeline Pilot (Accelrys Software: Pipeline Pilot. v.8.4), the protonation state of all compounds was adjusted to reflect the most prevalent form at a pH of 7.4. CORINA (Molecular Networks: CORINA. v.2.4) was used to convert these files to two-dimensional (3D) PDB coordinate files, which resulted in low energy 3D structures. The files were then processed with the python script prepare_ligand4.py, which comes with the Autodock Tools Suite (Morris et al., J Comput Chem. 2009; 30:2785-91). This script generates a pdbqt file, and adds partial charges to the ligand, sets all torsions in the ligand to active (to permit rotation), and merges all non-polar hydrogen atoms.

The DUSP5 PD structure (PDB code 2G6Z) was prepared for docking using the Autodock Tools Suite Grid maps were used in the energy calculations performed by Autodock. Partial charges were added and all non-polar hydrogen atoms were merged, resulting in a pdbqt file. The 13 different grid maps, one for each of the different atoms present in the chemical library of compounds (ex. C, H, F, Cl, etc.), were generated using Autogrid4. The grid box (the site used to dock the ligands) was positioned to cover the entire protein in a blind docking experiment, to ensure unbiased identification of binding location and orientation.

The docking parameter file (dpf), which contains the parameters that Autodock4 uses to dock ligands into the protein, was prepared using the python script prepare_dpf4.py, and default docking parameters were used, except that 50 separate docking calculations were performed with each calculation consisting of 1,750,000 energy evaluations and a root mean square deviation (rmsd) tolerance set to 2.0 angstroms (to define entry of structure into a given cluster). The dpf files were then automatically docked using the MUGrid Cluster (Marquette University) with HTCondor (Frey et al., Cluster Comput. 2002; 5:237-46; Litzkow et al., In Proceedings of the 8th International Conference of Distributed Computing Systems: 13-17 Jun. 1988; San Jose, Calif. 104-111) and AutoDock4 (Morris et al., J Comput Chem. 2009; 30:2785-91) using the Lamarckian genetic algorithm local search method, to perform the optimization of docking poses. The docking poses were then clustered on the basis of the rmsd between the coordinates of the atoms in a given ligand, and were ranked on the basis of calculated free energy of binding. The docking log files were then analyzed using the python script summarize_results4.py contained in the shell script sumresults_4.py (17), which rank orders all the dockings by binding energy. The results were then analyzed to find the best-clustered compounds with lowest free energy of binding as determined by Autodock4.2. Additional docking of all experimentally tested chemicals was performed as described above, but with 100 dockings trials.

Ligand-Based Searching.

As previously described, the CSD3 chemical library was electronically prepared and protonation state adjusted using Pipeline Pilot (15) Using OpenEye Scientific Software's Omega2 (21), three dimensional coordinates were calculated and stored in OpenEye Scientific Software's preferred file, .oeb.gz, for subsequent molecular overlay evaluation. OpenEye Scientific Software's Rapid Overlay of Chemical (ROCS) (22) matches a chemical query to a database of chemicals in search of hits. ROCS was used to rank-order hits based on overlay and optimal matching of electrostatics and molecular shape. ROCS was used to query using promising chemical hits from docking to identify related compounds from databases of Food and Drug Administration (FDA) approved drugs.

The ZINC library is a large database of commercially-available chemical compounds (the ZINC database; Irwin et al., J. Chem. Inf. Model. 52(7):1757-68 (2012); available at zinc.docking.org on the World Wide Web). Chemical structure information was obtained from the ZINC database as two-dimensional (2D) SDF files and prepared similarly to the CSD3 chemical library for use with OpenEye Scientific Software's ROCS. This further expanded the availability of chemical analogs available for experimental screening. ROCS calculations were also performed against the DrugBank (Wishart et al., Nucleic Acids Res. 2008; 36 Suppl 1:D901-6; Wishart et al., Nucleic Acids Res. 2006; 34 Suppl 1:D668-72) database of FDA approved drugs.

### Synthesis of RR505 and RR06. Synthesis of Carbazole-1,3,6-trisulfonic acid, Trisodium Salt (RR505)

Solid carbazole (3.0 g, 17.9 mmol) was placed in a 50-mL round-bottom flask and 67% H2SO4 (12 mL) was added dropwise at 22° C. and a slurry thus obtained was stirred and heated at 115±5° C. for 6 h. The resulting dark solution was cooled to room temperature and poured into a saturated NaCl solution (100 mL) containing NaOH (2.4 g, 60 mmol) to afford an ash-colored precipitate, which was filtered, washed with saturated NaCl solution (50 mL) and dried at 90° C. for 10 h to get 7.5 g of the crude product. The crude solid was dissolved in distilled/deionized water (150 mL), added activated charcoal Norit (1.1 g) and the resulting mixture refluxed for 15 min. The solution was filtered hot through a pad of celite, and evaporated slowly to afford a white powder of RR505 (5.5 g, 65% yield). 1H-NMR (400 MHz, D2O): 7.60 (1H, d, J=8.8 Hz), 7.79 (1H, d, J=8.8 Hz), 8.08 (1H, s), 8.53 (1H, s), 8.64 (1H, s), 1H-NMR (400 MHz, DMSO-d6): 7.58-7.66 (2H, m), 7.96 (1H, s), 8.24 (1H, s), 8.26 (1H, s), 10.81 (1H, s). 13C-NMR (100 MHz, D2O): 112.3, 118.8, 121.6, 121.64, 121.7, 124.5, 124.7, 125.5, 133.7, 134.7, 136.9, 142.1.

### Synthesis of Carbazole-1,3,6,8-tetrasulfonic acid, Tetrasodium Salt (RR506)

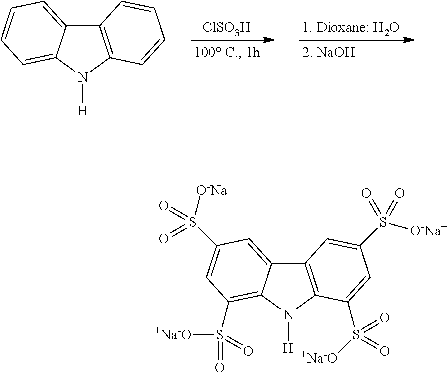

Solid carbazole (3.0 g, 17.9 mmol) was placed in a 50-mL round-bottom flask and chlorosulfonic acid (41.7 g, 358 mmol) was added in small portions with vigorous shaking at 22° C., after which the mixture was stirred and heated at 100±5° C. for 1 h. The resulting dark solution was cooled to room temperature and then poured slowly onto crushed ice (˜100 g). The resulting precipitate was filtered by gravity filtration and was dried by placing between paper towels. The resulting semi-dried solid was dissolved in ethyl acetate (150 mL), treated with Norit (1.5 g), and refluxed for 15 minutes and filtered hot through a pad of silica gel (˜1×1.8 inch). The filtrate was concentrated in vacuo and recrystallized from a 1:9 mixture of ethyl acetate and hexanes to afford a yellow solid, which was filtered and dried in vacuo. The dried solid was dissolved in a mixture of dioxane (20 mL) and distilled/deionized water (20 mL) and heated under reflux for 12 hours. The resulting solution was cooled to room temperature and was extracted with diethyl ether (2×50 mL) to remove nonpolar impurities. The aqueous layer was neutralized by a dropwise addition of NaOH solution (1 M) with continuous monitoring of pH using pH paper. The resulting solution was concentrated to ˜10 mL and acetone was added to afford a white powder of RR506 (3.5 g, 22% yield, average yield from 3 runs). 1H-NMR (400 MHz, D2O): 8.12 (2H, s), 8.68 (2H, s), 1H-NMR (400 MHz, DMSO-d6): 7.97 (2H, s), 8.26 (2H, s), 10.61 (1H, s); 13C-NMR (100 MHz, D2O): 121.9, 122.1, 123.8, 125.7, 134.7, 136.7.

### Alternative Synthesis of RR506

Solid carbazole (1.0 g, 6 mmol) and nitrobenzene (20 mL) were placed in a 50-mL round-bottom flask and chlorosulfonic acid (14 g, 120 mmol) was added in small portions at 22° C., after which the mixture was stirred at 22° C. for 72 h. The resulting solution was poured into aqueous saturated NaCl solution (100 mL) containing NaOH (0.96 g, 24 mmol) which resulted in a fluffy precipitate. The precipitate thus formed was filtered and dried. The solid was dissolved in distilled/deionized water (100 mL) and refluxed with 1.5 g of Norit for 15 min and filtered hot through a pad of celite. The filtrate was concentrated to ˜25 mL and RR506 was precipitated by addition of acetone. The precipitate was filtered and dried to afford RR506 as a white solid (2.9 g, 84% yield). 1H-NMR (400 MHz, D2O): 8.12 (2H, s), 8.68 (2H, s), 1H-NMR (400 MHz, DMSO-d6): 7.97 (2H, s), 8.26 (2H, s), 10.60 (1H, s).

Protein Production.

The DUSP5 PD gene was synthesized by Blue Heron (Bothell, Wash.) in both an active wild type form (DUSP5 PD-WT) and an inactive form, where the catalytic cysteine was mutated to a serine (DUSP5 PD-C263 S). The genes were inserted into Origene pEX plasmids with ampicillin resistance and an N-terminal hexa-histidine tag to facilitate protein purification. Plasmids were transformed into BL21(DE3) cells (Invitrogen) for expression.

For unlabeled DUSP5 PD(WT) preparation, an overnight culture was used to inoculate 2 L of LB (Luria-Bertani) media, with 50 μg/mL of ampicillin. Cells were grown at 37° C. to an OD600 of 0.7 and then induced with 0.6 mM isopropyl β-D-1-thiogalactopyranoside (IPTG) for 4 hours at 37° C., then for 14 h at 16° C. Cells were harvested using centrifugation and frozen prior to purification. Thawed cells were lysed in a buffer containing 50 mM Tris, 300 mM NaCl, 5 mM imidazole, and 10% glycerol at pH 7.8. Lysate was centrifuged at 15,000 rpm for 1 h The supernatant was loaded on to Ni-Sepharose Fast Flow resin (GE Healthcare) and washed with three times successively with 5 column volumes of lysis buffer containing 25 mM imidazole. Protein was eluted with lysis buffer containing 305 mM imidazole. Protein was then dialyzed in a buffer containing 50 mM potassium phosphate and 2 mM dithiothreitol (DTT) at pH 6.8.

For 15N-labeled DUSP5 PD(C263 S) preparation (for NMR titrations), an overnight culture was used to inoculate 2 L of LB media supplemented with 50 μg/mL of ampicillin. Cells were grown to an OD600 of 0.7 at 37° C., then harvested and washed with M9 minimal media (pH 7.0) (26). Cells were resuspended in 500 mL M9 minimal media containing 0.5 g 15NH4Cl, 2 g D-glucose, 5 mL Basal Medium Eagle with Earle's salts and sodium bicarbonate (Sigma Aldrich), 0.146 g L-glutamine (Sigma Aldrich) 1.0 mL 1M MgSO4, and 0.5 mL 1 M CaCl2 (pH 7.2). Additionally, a metal mix containing Zn, Mn, Cu, Co, B, and Mo salts was added to supply cells with necessary micronutrients (26). Cells were allowed to acclimate for 30 min at 37° C., then induced with 1 mM IPTG for an additional 4 h at 37° C. Cells were harvested and ¹⁵N-labeled protein was purified as described before with the addition of 2 mM dithiolthreitol (DTT) during all purifications steps.

p-Nitrophenolphosphatase (pNPP) Activity Assay.

To measure enzymatic activity of the DUSP5 PD and the inhibitory capacity of selected molecules, an in vitro phosphatase assay was developed based on previous studies (27). In this assay, DUSP5 PD dephosphorylates the substrate p-nitrophenol phosphate (pNPP, Sigma Aldrich), yielding p-nitrophenolate, which absorbs at 405 nm with an extinction coefficient of 18,000 M-1 cm-1.

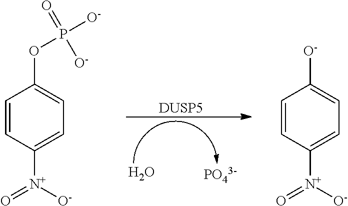

Thus, an increase in absorbance at 405 nm corresponds to the turnover of pNPP top-nitrophenolate. The assay was initially optimized in 1 mL quartz cuvettes, then was subsequently optimized for and validated in a 96-well plate format. All IC50 values were obtained using the 96-well plate assay format (see below). The assay buffer contained 100 mM Tris, 100 mM sodium chloride, 5 mM magnesium chloride, and 1 mM dithiothreitol (DTT) at pH 7.5. The pNPP substrate was prepared as a 50 mM stock by dissolving the solid substrate in assay buffer. The DUSP5 PD and pNPP were assayed initially in a cuvette (1 mL total volume) and initial velocities were fitted to the Michaelis-Menten equation (1)

$$v = \frac{{V}_{max} [ S ]}{{K}_{m} [ S ]} \qquad ( 1 )$$

where v is the initial velocity, Vmax is the maximum velocity, Km is the Michaelis constant, and [S] is the concentration of pNPP. Data were fitted using a nonlinear least squares fit to eq. 1, with GraphPad Prism 6 software.

Validation of pNPP Assay for High Throughput Screening (HTS).

For the 96-well plate validation assay, sodium orthovanadate (Sigma Aldrich) was utilized as a positive control for inhibition (Swarup et al., Biochem Biophys Res Commun. 1982; 107:1104-9.) at a final concentration of 10 μM, to completely block activity. All plate assays were performed in standard 96-well clear bottom plates (Thermo Scientific Nunc) with a total assay volume of 200 μL, using a SpectraMax M5 Microplate Reader (Molecular Devices). The plate validation assay was performed with replicate columns of positive control wells, negative control wells and blank wells. Blank columns contained only buffer and pNPP. Negative control (uninhibited) contained buffer, pNPP, and DUSP5 PD; and, positive control contained the same components, but also contained 10 μM sodium orthovanadate. The plate was then shaken and allowed to equilibrate in the spectrophotometer at 25° C. for 30 min. After incubation, 4 μL of a 1 μM enzyme stock was dispensed into appropriate wells utilizing a single-channel pipette. This produced a final enzyme concentration of 1 μM. Before a read was taken, the plate was shaken for five seconds. The initial rate for the DUSP5 PD reaction was linear for approximately 90 min; and, the plate was kept in the spectrophotometer at 25° C. for an additional 80 min after the kinetic read. The endpoint reading was subsequently taken at 90 min after initiation of reaction. Slopes from the kinetic read, as well as single-point absorbance values at the 90-minute endpoint read, were then averaged. For blank wells and positive control wells, both slope values (continuous assay) and single point absorbance values (fixed time assay) were approximately zero, as expected. Standard deviations were calculated and a Z′ value (Zhang et al., J Biomol Screening. 1999; 4:67-73) subsequently determined using the following equation:

$${Z}^{′} = 1 - \frac{3 ( {σ}_{p} + {σ}_{n} )}{\vert {μ}_{p} - {μ}_{n} \vert} \qquad ( 2 )$$

where σp is the standard deviation for the positive control, on is the standard deviation for the negative control, μp is the mean for the positive control, and μn is the mean for the negative control. The Z′ value is a coefficient denoting the quality of a high throughput screening assay, reflecting both the variation in data and dynamic range for the assay. A good assay exhibits a high signal to background ratio. A Z′-factor of 1.0 reflects an ideal assay; and, for an assay to be considered reliable, must exceed 0.5 (Zhang et al., J Biomol Screening. 1999; 4:67-73).

IC₅₀ Measurements.

IC₅₀ values were obtained using the assay described above, in 96 well plates. The maximum inhibitor concentration screened in any plate was 300 mM and the minimum was 1.5 μM. The IC₅₀ plate was designed so that the first column of wells served as blanks, with wells containing only buffer and substrate. The second column of wells functioned as the plate negative control, with each well containing buffer, substrate and enzyme. The remaining wells in the plate contained buffer, substrate, enzyme, and varying amounts of inhibitor, with inhibitor concentration increasing from left to right across the plate. Data points were collected in triplicate, and inhibitor concentrations were chosen to provide data equally spread on a logarithmic scale. The composition of buffer and the concentrations of substrate and enzyme utilized were identical to those in the plate validation assay. After initiation and shaking, a ten-minute kinetic read was taken.

For each plate assayed, the slope values for all negative control wells were averaged and the measured value considered representative of full enzymatic activity. Fractional activity was then calculated by dividing the slope of each inhibitor well by this value, determining the relative amount of enzyme activity observed at each concentration of inhibitor. Values were then plotted as percent activity versus the log of the concentration of inhibitor, and fitted to the following equation:

$$y = Bottom + \frac{( Top - Bottom )}{1 + {10}^{z - {logAZ}_{30}}} \qquad ( 3 )$$

where Top and Bottom are plateaus for the values of initial velocity when uninhibited and fully inhibited, respectively.

Nephelometry.

Nephelometry is a technique for measuring the relative aggregation of particles in solution, based on the light-scattering properties of molecular aggregates (30). To explore the ability of the chemicals studied herein to form aggregates, which can lead to artifactual inhibition effects, nephelometry was performed. Compounds were tested for aggregation in 96-well plates using a buffer containing 100 mM Tris base, 100 mM sodium chloride, and 5 mM magnesium chloride at pH 7.5. Each compound analyzed in these experiments contained concentrations of compound ranging from 10-100 μM, recorded in quadruplet. Each plate was analyzed at two separate gain values of 52 and 72. Data were collected using a BMG NEPHELOstar Plus, equipped with a 635 nm laser.

NMR Binding Assay.

NMR samples of DUSP5 PD(C263 S) were prepared for 2D 1H-15N HSQC (heteronuclear single quantum coherence) spectral titration studies. The 15N-labeled DUSP5 PD(C263) protein was concentrated using an Amicon Ultra-4 centrifugal device (Millipore) to 600 μM.

NMR samples were prepared with the following conditions for RR505: ±250 μM RR505, 250 μM DUSP5 PD (C263S), 10% D20, 50 mM potassium phosphate, 100 mM KCl, and 2 mM DTT at pH 6.8.

NMR samples were prepared with the following conditions for CSD3-2320: ±500 μM CSD3-2320, 500 μM DUSP5 PD (C263S), 10% D20, 50 mM potassium phosphate, 100 mM KCl, and 2 mM DTT at pH 6.8. NMR experiments were performed on a 500 MHz Varian NMR System using a triple resonance probe with z-axis gradients at 25° C.

Cell Culture and Western Blots.

Human Umbilical Vein Endothelial Cells (HUVECs) were maintained in EBM2 media (Lonza, CC-3156) supplemented with a combined serum and growth factor kit (Lonza, CC-4176). Cells were incubated with the indicated concentrations of drug for 2 h and lysed with RIPA buffer (Sigma, R0278) on ice. Cell lysates were cleared by centrifugation at 16,300×g for 30 min. The resulting supernatants were isolated and protein concentration determined by Lowry assay (DC protein assay, BioRad). 2× laemmli sample buffer was added to equal amounts of protein and separated by SDS-PAGE. The protein gels were transferred to polyvinylidene difluoride (PVDF) and immunoblotted using antibodies to phosphorylated ERK (Cell Signaling, #9106) and total ERK (Cell Signaling, #9102). Bound antibodies were visualized using horseradish peroxidase-linked anti-rabbit IgG, anti-mouse IgG (Cell Signaling Tech., #7074 and #7076) and ECL reagents (Thermo Scientific, 34080) according to the manufacturer's protocol.

ERK Dephosphorylation Assay.

For this assay, 10 ng of GST-tagged recombinant phosphorylated ERK2 (R&D Systems, 1230-KS) was incubated with and without the indicated DUSP5 proteins (0.5 nM final concentration) for 15 min at room temperature, with or without the indicated drugs. The reactions were halted with 2× Laemmli sample buffer and subjected to SDS-PAGE. The proteins were transferred to polyvinylidene difluoride (PVDF) and immunoblotted using antibodies to pERK (Cell Signaling Tech., #9106) and Total ERK (Cell Signaling Tech., #9102). Bound antibodies were visualized using horseradish peroxidase-linked anti-mouse IgG (Cell Signaling Tech, #7076S) and anti-rabbit IgG (Cell Signaling Tech, #7074S), respectively, and ECL reagents (Pierce, 34708) according to the manufacturer's protocol. IC50s were calculated using GraphPad Prism 6 software.

Docking and Ligand-Based in Silico Searches Yield Candidate Small Molecules that Target DUSP5 PD Domain.

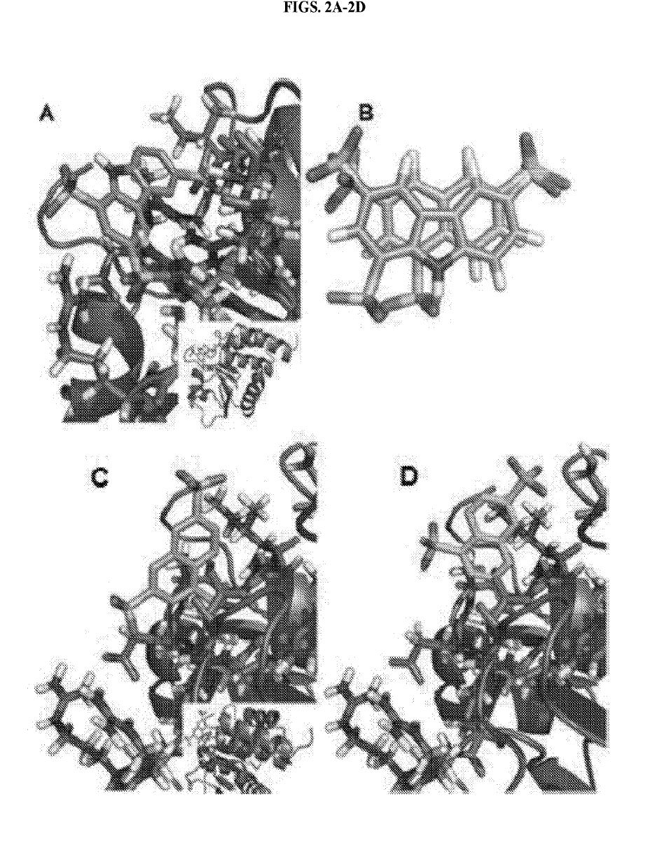

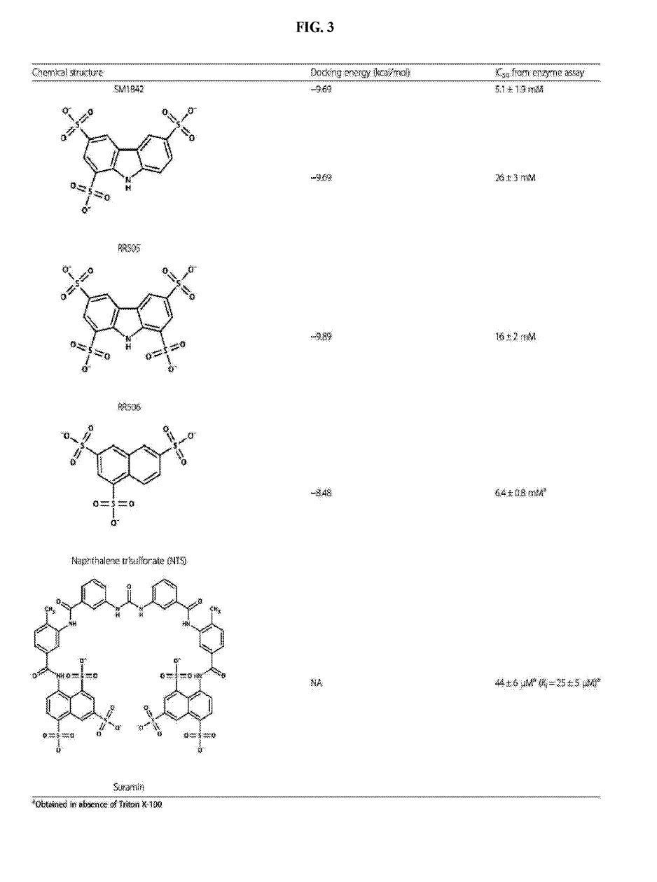

Docking of the CSD3 in-house collection of 11,500 chemicals produced a rank-ordered list of compounds that were tested using the phosphatase assay (discussed below). One promising compound, SM1842, displayed attributes associated with lead-like chemicals (31). The docking pose from the lowest energy cluster for SM1842, with calculated binding energy of −9.69 kcal/mol, is shown in FIGS. 2A-2D. 1H NMR analysis of the commercially sourced SM1842 sample did not match the expected 1H NMR spectrum, so this compound was resynthesized and spectra were compared between commercial and resynthesized versions. The resynthesized compound, RR505 (FIG. 3), displayed the expected 1H NMR spectrum for the trisulfonated carbazole. Additional synthesis and 1H NMR analysis of a tetrasulfonated carbazole (i.e., an additional sulfonate, relative to SM1842), RR506 (FIG. 3), demonstrated that the commercial SM1842 sample was likely a mixture of RR505 and RR506, the tri- and tetrasulfonated carbazoles. Further experimental analysis focused on the single-component pure samples, RR505 and RR506.

Using the chemical structure of RR505 as a search template, additional chemical libraries were computationally screened to identify related structures that could also be tested.

One such compound, naphthalene trisulfonate, was identified from the in-house collection of chemicals and from the ZINC collection (23) of commercially available chemicals. This compound was identified using ROCS (OpenEye Scientific Software), which matches chemical queries to compounds in chemical libraries based on molecular shape and electrostatic properties. FIG. 2B shows the overlay of RR505 and NTS, using ROCS. NTS docked similarly to SM1842/RR505 in the DUSP5 PD active site pocket (FIG. 2A). Interestingly, the lowest energy poses for NTS and RR505 show a flipped binding mode relative to each other (FIGS. 2C and D). We hypothesized based on the ROCS alignment (FIG. 2B) that the docking algorithm would position the ligands similarly. While this is not the case for the lowest energy cluster, it is the case for the second lowest energy cluster. Similarly, RR506 was flipped relative to the lowest energy cluster pose of RR505 (not shown). And again, the second lowest energy cluster pose matched that of the lowest energy pose of RR505.

DUSP5 PD Domain Protein Generation, and Activity Determination—

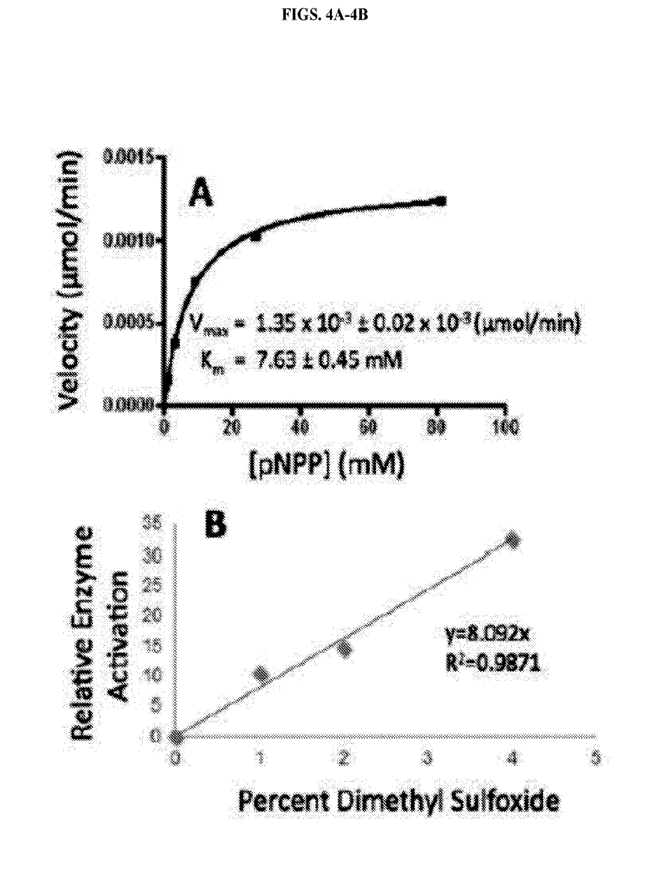

DUSP5 PD(WT) protein was used in enzymatic pNPP assays to determine whether compounds identified via docking and ligand-based methods bind, and with what affinity. To facilitate these assays, protein was expressed in E. coli and purified. To enable NMR titration studies (i.e., direct binding assays), DUSP5 PD(C263 S) was expressed in E. coli in M9 minimal media (15N labeled), and purified. SDS-PAGE gel analysis of DUSP5 PD(C263S) indicates >95% purity. The 2D 1H-15N HSQC NMR spectrum for a sample of the DUSP5 PD(C263 S) protein indicates good chemical shift dispersion, suitable for NMR titration experiments (vide infra), and indicating the protein is well-folded. To assess the ability of the compounds identified via docking to inhibit DUSP5 activity, a phosphatase assay was developed based on a previously published assay (27, 34). The substrate, p-nitrophenol phosphate (pNPP), has been shown to react with a wide variety of phosphatases. The assay was performed in 1 mL quartz cuvettes, at various substrate concentrations, and initial velocities were measured. Data were fitted to the Michaelis-Menten equation (FIG. 4A), yielding a Vmax of 1.35±0.02×10-3 (μmol/min) and a Km of 7.63±0.45 mM. Since some of the inhibitors to be screened were dissolved in DMSO, the effect of 1%, 2%, and 4% DMSO was investigated by substituting appropriate quantities of DMSO for some of the buffer mixture. Relative rates with and without DMSO were compared and plotted in FIG. 4B. DMSO appears to activate the DUSP5 PD reaction, consistent with a previous report for DUSP6 (33). Thus, assays done on compounds dissolved in DMSO must be performed such that DMSO concentration is kept constant, to ensure consistent results.

To demonstrate how the DUSP5 PD assay could be used to identify inhibitors, and measure IC50 values, a control inhibitor was used. The assay was performed with the addition of a known broad-spectrum inhibitor of phosphatases, sodium orthovanadate (vanadate) (28). Initial experiments were performed in 1 mL cuvettes and concentrations of vanadate were varied. Initial velocities from kinetic reads were plotted as a function of the log of vanadate concentration and fitted to equation 3, to obtain the IC50 of 8±8 nM.

Development of HTS Assay for Screening Inhibitors and Validation—

The pNPP phosphatase assay was also performed in a plate format to increase throughput by which inhibitors could be screened. To validate the plate assay, the Z′ factor (29) was determined using the plate arrangement described above. The absorbance values for the end-point assays and the slopes for the kinetic assays were averaged (summarized in Table 2) and Z′ values were calculated using equation 2. Both the end point assay (Z′=0.73) and the kinetic assay (Z′=0.74) formats resulted in Z′ factors in the acceptable range for an HTS assay.

TABLE 2
DUSP5 PD(WT) pNPP Enzymatic Assay Data for Z′ Calculation

| | |
|---|---|
| Positive control | Negative Control |

(a) End-point assays after 90 min.

| | | |
|---|---|---|
| Mean | 0.006 | 0.269 |
| Standard deviation | 0.004 | 0.02 |

(b) Kinetic (continuous) assay over 5 min.

| | | |
|---|---|---|
| Mean | −0.035 | 3.28 |
| Standard deviation | 0.091 | 0.193 |

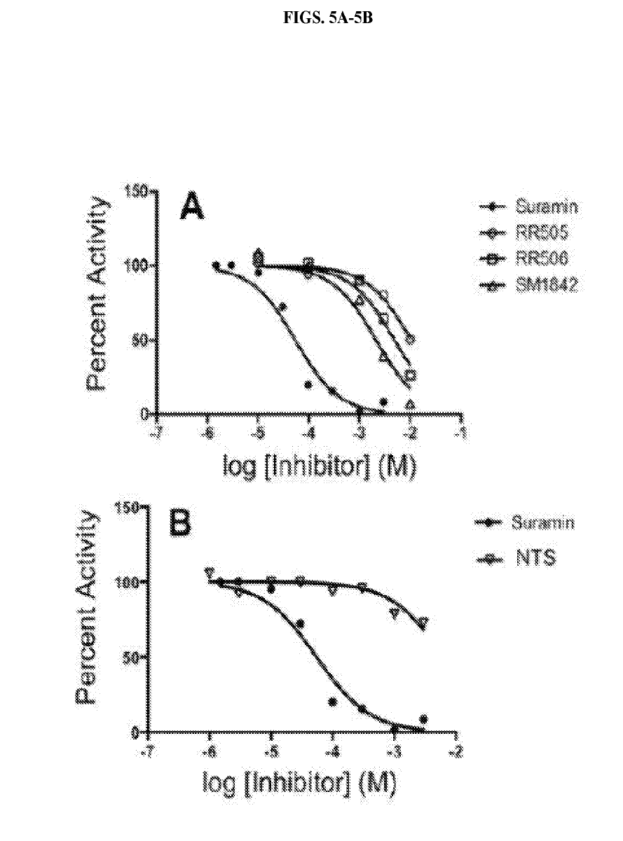

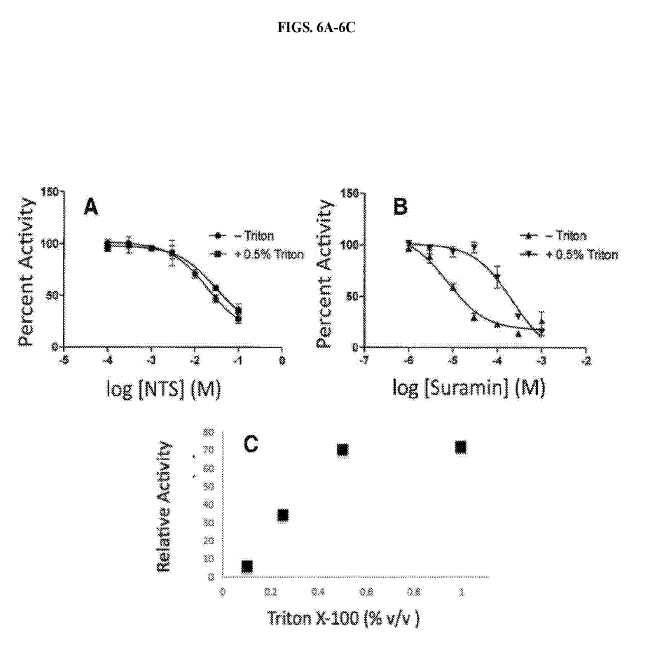

Compounds identified by protein-based (docking) and ligand-based (ROCS overlays) in silico screening methods were tested experimentally using the plate assay described above. Initial velocities were measured for the first 10 minutes of reaction, plotted (FIGS. 5A-5B), and then fitted to equation 3 to obtain IC50 values (summarized in FIG. 3). Note that SM1842 is a mixture of two components, likely RR505 and RR506. Triton X-100 was used in this assay (at 0.1%) to disrupt any small molecule aggregates that could be formed (FIGS. 6A-6C). This aggregation phenomenon is studied in depth later.

Suramin, an FDA Approved Analog, was Identified Via Lead Hopping—

Drugbank (Wishart et al., Nucleic Acids Res. 2008; 36 Suppl 1:D901-6; Wishart et al., Nucleic Acids Res. 2006; 34 Suppl 1:D668-72), a chemical library containing structures of FDA approved drugs, was also searched using ROCS, querying with the RR505 and NTS structures. Suramin was found as a match to the NTS structure. Indeed, Suramin (Table 1) is a superstructure of NTS, is comprised of two NTS substructures connected via a rigid linker, and is an FDA approved drug that is used to treat African sleeping sickness.

When Suramin was included in the pNPP phosphatase assay with DUSP5 PD (WT), it was one of the more potent inhibitors (FIG. 5). To further elucidate mechanism of inhibition, initial velocity inhibition profiles of Suramin were obtained, by measuring initial velocities at varied concentrations of substrate (pNPP) and inhibitor (Suramin). The inhibition profile fit best to the equation for competitive inhibition (eq. 4).

$$v = \frac{{V}_{□} [ S ]}{{K}_{m} ( 1 + \frac{[ I ]}{{K}_{i}} ) + [ S ]} \qquad ( 4 )$$

where v is the initial velocity, Vmax is the maximum velocity, Km is the Michaelis constant, and [S] is the concentration of pNPP. The data were fitted to the equation for competitive inhibition, to give a Ki of 24.6±5.2 μM. Competitive inhibition suggests that inhibition occurs via specific blockage of the phosphatase active site (Suramin competes for pNPP binding in the phosphatase active site).

Confirmation of Inhibitor(s) Binding to DUSP5PD Using NMR—

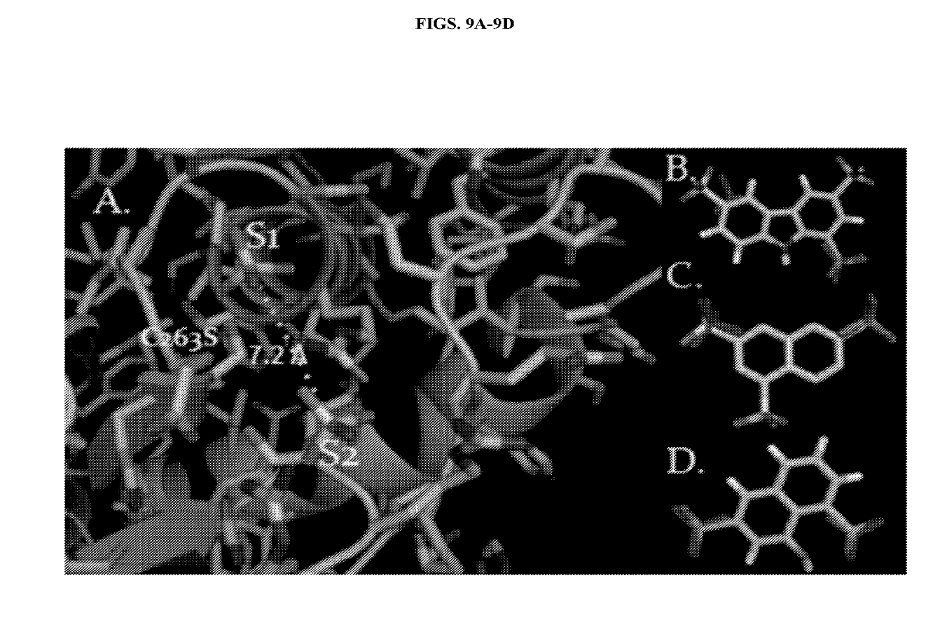

Screening projects typically require a secondary assay that confirms binding via an alternative mechanism, relative to the primary assay, to rule out false positive inhibition measurements. To this end, NMR titrations were performed with RR505 and CSD3-2320, measuring changes in 2D 1H-15N HSQC (heteronuclear single quantum coherence) crosspeaks for DUSP5 PD(C263 S), due to inhibitor binding. HSQC NMR spectra for DUSP5 PD(C263 S) obtained in the absence and presence of RR505 inhibitor show exchange broadening of crosspeaks, consistent with direct binding of RR505 to DUSP5. The corresponding HSQC spectral overlay for CSD3-2320 revealed more dramatic chemical shift changes (FIG. 9C), also consistent with direct binding. The visibility of black crosspeaks is likely due to exchange broadening (or chemical shift change) of the amides corresponding to the missing red crosspeaks, consistent with direct binding of RR505 and CSD3-2320 to DUSP5 PD.

Suramin Shows the Most Potent In Vitro Effects in Endothelial Cells.

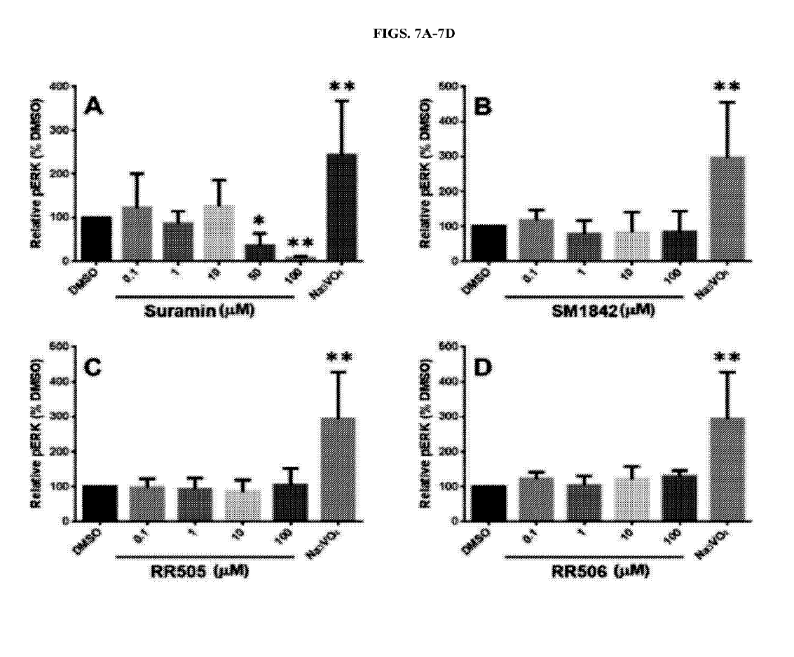

While in vitro binding and enzyme inhibition studies provide a useful characterization of lead molecules, cell-based assays provide a more reliable predictor of efficacy in vivo. To this end, ability of DUSP5 ligands to alter levels of pERK in HUVEC cells was monitored. But, these HUVEC cellular assays do not show biologically useful levels of potency for the sulfonated inhibitors RR505 and RR506 (FIG. 7), consistent with enzyme inhibition showing IC₅₀'s in the mM range (FIG. 3). HUVEC cellular studies with Suramin show an effect in cells (decreased pERK levels) that is opposite to that which was expected for DUSP5 inhibitors (FIG. 7). This effect is probably due to Suramin binding to other protein(s) in the VEGF pathway (40) besides or in addition to DUSP5. Control inhibition with vanadate gave the expected increase in pERK levels due to DUSP5 inhibition. Thus, Suramin's cellular mechanism is complex, and involves effects other than simply inhibiting DUSP5 activity. For these reasons, and those discussed above, Suramin is not a viable lead molecule for DUSP5-based therapeutic interventions; at least not in its current form.

Suramin Aggregates but NTS does not.

To investigate whether Suramin aggregates in solution, a property often investigated with small molecules, we performed detergent treatment assays. Triton X-100 is a detergent that is able to break up small molecule aggregates, while not significantly interfering with the assay (35). NTS and Suramin were compared using DUSP5 PD IC₅₀ assays performed with and without detergent. FIGS. 6A-6C show that Suramin inhibition is decreased by detergent, while NTS inhibition is not affected, suggesting that at least some of Suramin's inhibition is due to aggregation. We concluded that Suramin forms aggregates in vitro which can lead to non-specific protein inhibition effects. Beyond any adverse implications for repurposing Suramin in this project, the aggregation phenomenon raises more global concerns regarding the current clinical use of Suramin, and may in part explain some of the known toxicity associated with Suramin (32). Indeed, literature on Suramin (37, 38) indicates that it can bind to many protein targets, so may lack specificity in its mechanism of inhibition. Thus, while initially promising, Suramin does not exhibit properties of a good drug lead molecule (even though it is FDA approved).

Modeling Studies Determine Additional Potential NTS (Suramin) Binding Sites Outside of the Active Site on DUSP5 PD.

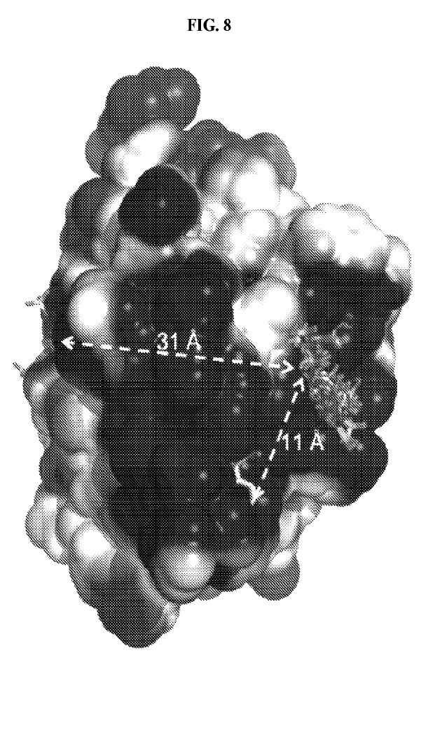

The above-mentioned competitive inhibition of Suramin versus pNPP binding suggests that—while Suramin may aggregate it can also competitively block the phosphatase active site, and apparently with higher affinity than NTS. Since Suramin is effectively a tethered NTS, it is possible that there is an additional NTS binding site outside of the phosphatase active site pocket that yields higher affinity due to bivalent (tethered) NTS binding. To explore this hypothesis, docking of NTS outside of the active site pocket (i.e., “external binding”) was performed. In FIG. 8, all 100 docking poses of external binding NTS are displayed on an electrostatically colored map of DUSP5 PD, revealing that there are only three general areas in which the NTS is predicted to bind; and, the largest cluster is in the active site pocket, as expected. The main secondary external binding pocket is 31 Å away from the active site pocket, while another secondary pocket is 11 Å away; but, this site lacks any significant density of positively charged sidechains, as would be needed for binding the negatively-charged NTS. No binding pocket was found closer than 11 Å (FIG. 8), which is further than the distance between the two NTS substructures in Suramin. The linker is sufficiently rigid as to prevent optimal alignment for binding both NTS substructures to the active site, and either of these potential secondary external binding pockets. Thus, if the two NTS molecules contained in Suramin are both binding to DUSP5 PD pockets, it might rather be via two separate DUSP5 PD molecules, as in a DUSP5 PD dimer or higher order aggregate. The latter possibility would be consistent with the observed ability of Suramin to cause aggregation of DUSP5 PD (in NMR 2D 1H-15N HSQC titrations; data not shown). The former possibility is consistent with literature reports that DUSP5 forms dimers (Zhang et al., J Biomol Screening. 1999; 4:67-73). Future studies will be directed to distinguishing these possibilities; but, it is clear that the Suramin inhibition mechanism is complex, and includes a combination of aggregation (of Suramin and of perhaps also of DUSP5 PD) as well as direct blockage of the phosphatase active site, based on the observation of competitive inhibition. Furthermore, cellular assays to monitor relative pERK2 levels in HUVEC cells indicate that Suramin decreases pERK levels (see discussion below), rather than increase as would be expected for a DUSP5 inhibitor like vanadate.

Future Directions with Suramin.

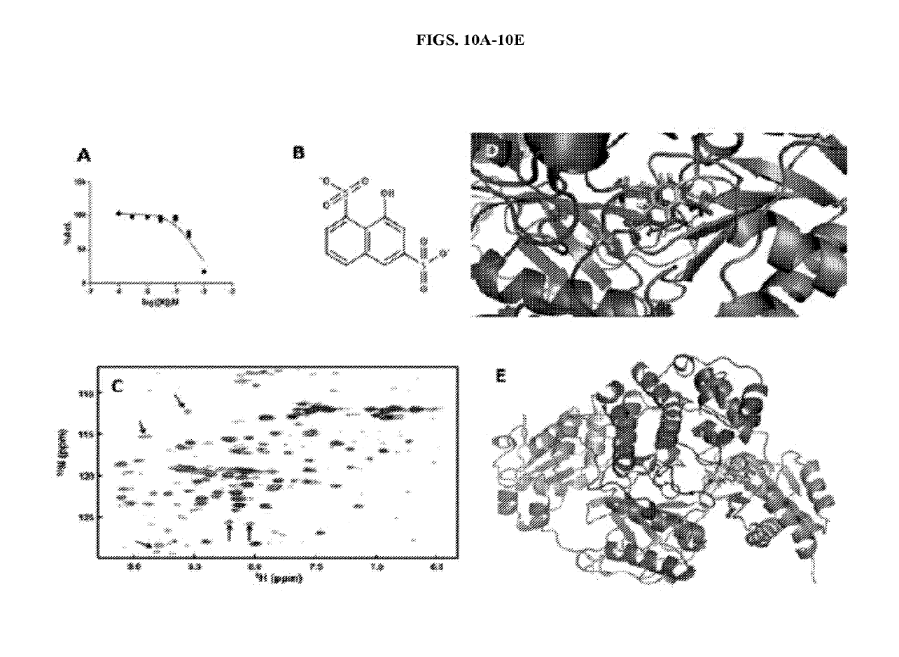

The ability of Suramin to act as a competitive inhibitor indicates that Suramin (i.e., the NTS group of Suramin) can bind in the phosphatase active site and—importantly—that the linker attached to NTS, in making Suramin, does not disrupt DUSP5 binding to the NTS fragment. Thus, future studies could be directed toward using NTS with a linker attachment like that in Suramin, but with different chemical composition in the linker, and tethered to a different fragment [NTS-(linker)-(new fragment)], such that inhibition occurs without any aggregation artifacts. This new fragment can be identified using NMR fragment-based screening methods, which are now possible since we have obtained high quality 2D 1H-15N HSQC (heteronuclear single quantum coherence) spectra (FIG. 10C). Such methods, as well as docking and ligand-based lead hopping (e.g., using ROCS), and traditional medicinal chemistry (e.g., bioisosteric replacements), will be used to design improved analogs of our initial sulfonated lead molecules, NTS, RR505, and R506.

Distance Between Sulfonates in the DUSP5 PD Active Site Identifies a Novel Small Molecule with Greater Potency.

While protein-based methods (i.e., docking) have identified a series of weak binding polysulfonated lead molecules (FIG. 3), and lead-hopping with ROCS has identified the FDA-approved drug Suramin, none of these are viable drug leads or in vivo pharmacology probes without further modification. An interesting feature of all these weak-binding lead molecules is the presence of at least two charged sulfonates, separated by 6-9 Å. This led us to hypothesize that this trend is occurring because the active site pocket of DUSP5 PD binds a peptide loop from ERK2 containing two phosphates, so is designed to accommodate two negatively charged functionalities separated by this approximate distance. Indeed, DUSP5 PD was found to crystallize with two sulfate anions bound, at an S—S distance of 7.2 Å (FIG. 9A).

These observations led us to conclude that the key pharmacophore feature for DUSP PD binding is two negatively charged groups (such as sulfates or sulfonates; possibly also carboxylates, tetrazoles and sulfonamides), tethered by a core scaffold (e.g., carbazole, naphthalenes). Recognizing this common theme amongst ligands, and inspection of the active site pocket of the crystal structure of the human DUSP5 PD (12), led us to a hypothesis regarding the required pharmacophore features of DUSP5 PD ligands. Since the DUSP5 PD crystal structure contains two sulfate ions in the active site pocket (FIG. 9A) in the regions suggested to be occupied by the di-phosphorylated substrate (pThr-Glu-pTyr, of ERK2), and our ligands generally possessed at least two sulfonates (FIG. 3), we reasoned that two such negatively charged moieties—appropriately positioned—are a necessary feature of any DUSP5 PD inhibitor.

To this end, a ligand-based search strategy based on the two bound sulfate ions was then pursued. These sulfate ions are positioned 7.2 Å from each other (FIG. 9A), with one located where the phosphate to be cleaved would reside (this site is called S1), proximal to the Cys263 nucleophile (FIG. 9A). The other, termed S2, is located 7.2 Å away, in an arginine-rich pocket. Overlay of RR505 with the S1 and S2 sulfates was less than optimal (FIGS. 9B-9C), while the NTS overlay was better (FIG. 9D).

Using this pharmacophore feature in a ligand-based screen, a naphthalene-based disulfonate compound, CSD3-2320 (also known as CSDDD2320), was identified (FIG. 10B). CSD3-2320 had an IC₅₀ of only 33 mM if assayed using the phosphatase domain alone (FIG. 10A), but 33 micromolar (μM) if assayed using the full-length DUSP5 with ERK2 as substrate. The 1D 1H NMR spectrum confirms that CSD3-2320 is pure and matches the expected structure. NMR HSQC titration experiments (FIG. 10C) confirm direct binding to DUSP5 PD, with several crosspeaks being shifted in the presence of CSD3-2320.

When CSD3-2320 is positioned in the model of the ERK2-DUSP5 complex (FIGS. 1A-1B) by overlaying the sulfonates on the phosphate groups of the ERK2 activation loop, a close superposition is obtained that positions the phenolic ring of the CSD3-2320 naphthalene core directly where they tyrosine would bind (FIGS. 10D-10E). Furthermore, this positioning placed the phenolic hydroxyl group in a position that could mimic a water nucleophile that would attack phosphorylated enzyme in a subsequent step. Thus, CSD3-2320 has features that may be generally useful for inhibiting dual specificity phosphatases that bind dual-phosphate containing substrate proteins that contain a pThr-X-pTyr motif, where pTyr is the first to be dephosphorylated.

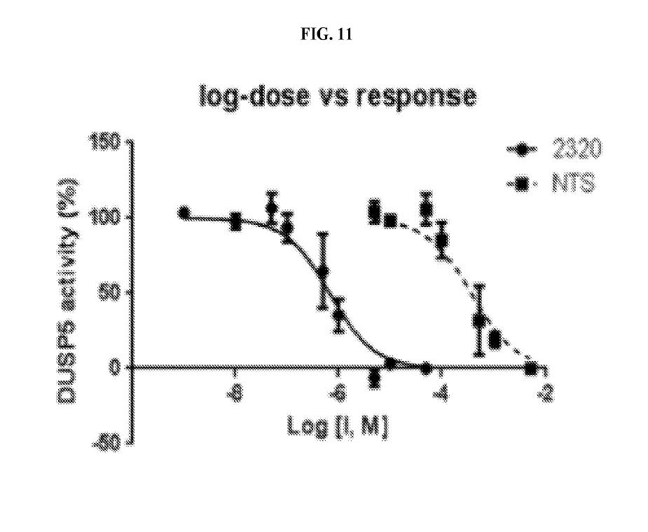

We then compared the inhibitory activity of NTS and CSD3-2320 in the more biologically relevant DUSP5 assay using full-length DUSP5 and the native substrate pERK (FIG. 11). The IC₅₀ values for NTS and CSD3-2320 are 221±87 μM and 538±271 nM respectively, in this DUSP5/pERK assay. A 1,000 fold increase in IC₅₀ activity is observed for CSD3-2320 compared to NTS in this assay. Interestingly, the inhibitory activity of CSD3-2320 towards GST-tagged full length DUSP5, when pNPP was used as a substrate was significantly weaker than with pERK as substrate, giving an IC₅₀ of 563±177 μM.

Importantly, nephelometry measurements indicate that CSD3-2320 has no propensity to aggregate. Thus, the 7.0-7.5 Å—separated disulfonate is a validated pharmacophore feature for inhibition of the DUSP PD. And, CSD3-2320 is our most potent inhibitor of DUSP5 PD activity, and a viable chemical genetic probe and drug lead molecule for inhibition of DUSP5 activity.

CSD3-2320 Inhibition in a Biologically Relevant Assay with Native Substrate and Enzyme.

While CSD3-2320 shows good inhibition in the DUSP5 PD assay using pNPP as substrate, this assay lacks full biological relevance, because the full length DUSP protein also contains an ERK binding domain, tethered via a flexible linker (FIG. 1A). And, the native substrate for DUSP5 is the pERK2 protein, which is much larger and capable of a more complicated range of inter-molecular interactions that the pNPP substrate (which is intended only to mimic the phospho-tyrosine of pERK). Thus, while the DUSP5 PD/pNPP assay is an effective preliminary screen (with direct binding confirmed using NMR-based titrations), a subsequent assay using full length DUSP5 and pERK2 substrate will provide the ultimate in vitro assessment of potency for a lead molecule. To this end, an assay was development using ERK2 and full-length DUSP5, monitoring pERK levels in a western blot, as a function of pERK2 substrate concentration. Using this DUSP5/pERK2 assay, IC50 values for CSD3-2320 and NTS were found to be 538+271 nM and 221+87 μM, respectively. This surprising increase in potency for CSD3-2320 (but not NTS) with full length DUSP5 underscores the importance of performing secondary assays with biologically relevant forms of enzyme and substrate.

As a control experiment, CSD3-2320 inhibition of pNPP activity was measured using full length DUSP5 (in a hybrid version of our two assay conditions), and the IC₅₀ value was found to be 561+177 μM, similar to the value observed in the pNPP assay with DUSP5 PD (484+80 μM). Thus, the increased potency for CSD3-2320 in the full length DUSP5/pERK2 assay is a function of using the ERK2 as substrate for full-length DUSP5, rather than pNPP. While this strong dependence of inhibition on the presence of the ERK binding domain (EBD) and native substrate (pERK) may be surprising, since inhibitor is simply blocking the active site of the phosphatase domain, it is perhaps consistent with the fact that a clinically observed S147P mutation—also remote from the phosphatase domain (see FIG. 10E)—has an effect on biological activity of DUSP5. Such interaction between remote domains in DUSP5 clearly has mechanistic and clinical relevance, and will be the topic of ongoing studies.

In conclusion, the potency of CSD3-2320 (but not NTS) under the more biologically relevant conditions of the DUSP5/pERK2 assay is in a range that is suitable for a chemical genetic probe and for a drug lead. Based on results with Suramin and NTS, there may be utility in tethering two CSD3-2320-like fragments to achieve even greater potency; but, this would need to be done in such a way as to avoid the nonspecific aggregation effects that were observed in Suramin. The unique molecular features of CSD3-2320—two sulfonate mimics of phosphate groups separated by 7.2 Å, on a rigid chemical platform, and a phenolic ring to mimic tyrosine—may make it useful as a probe for other dual specificity phosphates that bind substrates containing a pThr-X-pTyr motif. Finally, since inhibition potency is greatest using full-length protein with pERK substrate, there appears to be domain-domain (EBD-PD) communication in DUSP5 that affects the phosphatase domain active site. This has relevance for the clinically observed S147P mutation, which is observed in a remote region of DUSP5 (FIG. 10E), relative to the phosphatase domain.

### Example 2—Profiling Compound CSDDD2320 Against Phosphatase Targets

Assays were performed to profile compound CSDDD2320 against 21 phosphatase targets at 1 concentration (30 μM) in triplicate measurements. The aim was to identify whether the compound can affect the activity of the various phosphatase targets selected.

Results:

The results observed as % activity change compared to control are presented in Table 2. Intra-assay variability was determined to be less than 10%. Inhibition of target activity by the compound gives negative (−) values while activation of target activity gives positive (+) value. Values of >10% change in activity compared to control are generally considered to be significant.

The profiling data for the compound CSDDD2320 against the various phosphatase targets showed weak to moderate inhibition with some of the phosphatase targets (Table 3). Table 3 presents percent activity change of various phosphatase targets in the presence of a compound CSDDD2320, arranged from the greatest inhibition (− value) to activation (+ value). Phosphatases DUSP22 (MKPX), PTPRC (CD45), and PTPRE all showed inhibition of greater than 10% with the compound CSDDD2320. The phosphatase target DUSP22 (MKPX) showed the highest inhibition by the compound with an inhibition (compared to the control) of −13%. The remaining phosphatases tested had either no or low inhibition rates (ranging from 0% to −7% compared to the control) or gave a slight activation with the addition of the compound (ranging from 2 to 7% above the control).

TABLE 3
Percent Activity Change

| Number | Phosphatase Target | % Change |
|---|---|---|
| 1 | DUSP22 (MKPX) | −13% |
| 2 | PTPRC (CD45) | −12% |
| 3 | PTPRE | −11% |
| 4 | PP2Ca | −7% |
| 5 | PTPRS (PTP-sigma) | −6% |
| 6 | PTPN6 (SHP1) | −5% |
| 7 | PTPN1 (PTP1B) | −4% |
| 8 | PP1A | −3% |
| 9 | PP2A | −3% |
| 10 | PTPN12 (PTP-PEST) | −1% |
| 11 | PP1B | −1% |
| 12 | PTPRF (LAR) | 0% |
| 13 | CDC25B | 0% |
| 14 | PP2Cg | 0% |
| 15 | PTPN2 (TC-PTP) | 2% |
| 16 | CDC25C | 4% |
| 17 | PTPN11 (SHP2) | 4% |
| 18 | PTPRA (LRP) | 5% |
| 19 | PTPN13 (FAP-1) | 6% |
| 20 | CDC25A | 7% |
| 21 | PTPN7 (LC-PTP) | 7% |

The compound CSDDD2320 showed inhibition in 11 out of 21 phosphatase targets (ranging from −1% up to −13% compared to the control) with the phosphatases DUSP22 (MKPX), PTPRC (CD45), and PTPRE showing the greatest inhibition by the compound CSDDD2320 with changes of −13%, −12%, and −11%, respectively. The other 8 phosphatases (PP2Ca, PTPRS (PTP-sigma), PTPN6 (SHP1), PTPN1 (PTP1B), PP1A, PP2A, PTPN12 (PTP-PEST), PP1b) had low inhibitions of between −1% to −7%. Three of the phosphatases show no changes, including PTPRF (LAR), CDC25B, and PP2Cg, and the remaining 7 phosphatases gave slight activations with the addition of the compound. At a testing concentration of 30 μM, the strongest inhibition observed was 13%, against DUSP22 (MKPX). These data indicate that, compound CSDDD2320 is a fairly specific phosphatase inhibitor.

Methodology

Protein Phosphatase Assay: The DUSP22, PTPN2 (TC-PTP), PTPN7 (LC-PTP), PTPRA (LRP), PTPRC (CD45), PTPRE, PTPRS, PTPN1(PTP1B) and PTPN13(FAP-1) phosphatase activity was determined by performing assays at 37° C. for 15 minutes in a final volume of 100 μl according to the following assay reaction recipe:

Component 1. 5 μl of diluted active protein phosphatase

Component 2. 50 μl of 1.5 mM pNPP substrate assay solution

Component 3. 25 μl of phosphatase assay buffer

Component 4. 20 μl of compound (150 μM) or 10% DMSO

The assay was started by incubating the reaction mixture at 37° C. for 15 minutes. After the 15 minute incubation period, the assay was terminated by the addition of 25 μl of 2N NaOH stopping solution. The absorbance of the reaction solution was measured in a spectrophotometer at 405 nm. Blank control was set up that included all the assay components except the addition of the pNPP substrate (replaced with equal volume of phosphatase assay buffer). The corrected activity for various phosphatase targets was determined by removing the blank control value.

The PTPN6 (SHP1), PTPRF (LAR), PP1A, PP1B, PP2A, PTPN12(PTP-PEST), PTPN11(SHP2), CDC25A, CDC25B, CDC25C, PP2Ca, and PP2Cg phosphatase activity was determined by performing assays at 37° C. for 15 minutes in a final volume of 50 μl according to the following assay reaction recipe:

Component 1. 10 μl of diluted active protein phosphatase

Component 2. 5 μl of 1 mM tyrosine phosphopeptide-2 substrate (for PTPN6, PTPRF, PTPN12, and PTPN11) or threonine phosphopeptide substrate (for PP1A, PP1B, PP2A and PP2Cg) or PI(3,4,5)P3 substrate (for PTEN) or OMFP substrate (for CDC25A, CDC25B and CDC25C)

Component 3. 25 μl of phosphatase assay buffer

Component 4. 10 μl of compound (150 μM) or 10% DMSO

The assay was started by incubating the reaction mixture at 37° C. for 15 minutes. After the 15 minutes incubation period, the assay was terminated by the addition of 100 μl of Biomol Green reagent. The absorbance of the reaction solution was measured in a spectrophotometer at 650 nm. Blank control was set up that included all the assay components except the addition of the tyrosine phosphopeptide-2 substrate or related substrate (replace with equal volume of phosphatase assay buffer). The corrected activity for various phosphatase targets was determined by removing the blank control value.

### Example 3—Phosphatase Inhibitory Activity of RR535, RR601, and RR701

A NCI compound library was screened which revealed NCI-2602 as a compound with phosphatase inhibitory activity for phosphatases SHP-1 and PTP1B. NCI-2602 was resynthesized as RR535. RR535 is chemically distinct from SM-2320 and SM-1842. RR535 was in turn dimerized to form RR601 (a novel small molecule). The RR535 monomer is active in the 1 nM range while the RR601 dimer is active in the 30 mM range. Unlike other non-specific phosphatase inhibitors like Suramin, RR535 and RR601A do not aggregate. This non-aggregating property is a key distinction.

A published compound NSC-87877, which is a dimer comprised of two distinct molecules, has also been identified as a phosphatase inhibitor in the 1.7 mM range. Although there are some similarities between NSC-87877 and RR601, they are structurally very distinct entities.

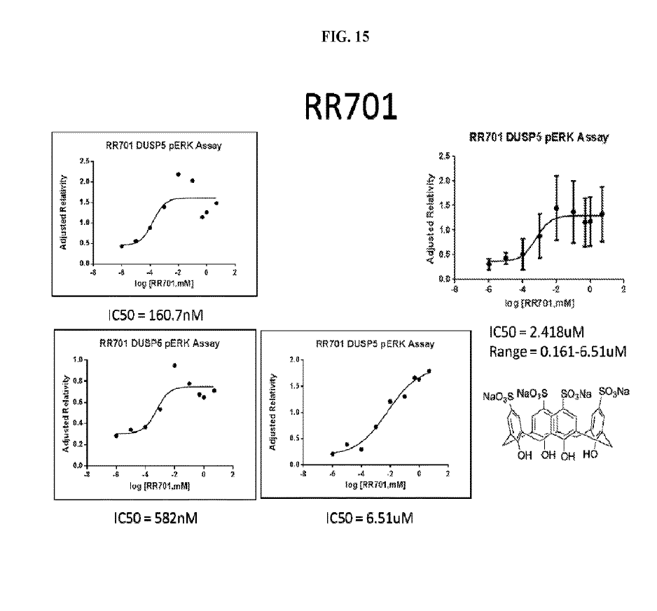

Compound RR701 was synthesized based on the thesis that DUSP5 antagonists comprising of two negatively charged sulfonate groups tethered by a core scaffold that preserves the separation of about 7 Å between them, where flexibility in the scaffold provides for this “separation of 7 Å” characteristic. A core scaffold of biphenyl and its planar analogs (e.g., carbazole), naphthalene, and conformationally-adaptable calixarene motifs were chosen. The conformational-adaptability of calixarene framework is the rationale for the base structure of RR701. Indeed, RR701 shows robust activity in the DUSP5:pERK western blot assay (FIG. 15).

### Example 4—Global Kinase and Phosphatase Profiles

To develop phosphates inhibition profiles, the SM1842 compound was tested at 7.5 μM. A kinase screen was performed using 352 protein kinases. Only 1 kinase was inhibited by 82% relative to control. 35 kinases exhibited inhibition between 15% to 55% relative to control. Out of 352 protein kinases screened, only one kinase was activated over 38% relative to control. Eighteen (18) kinases were activated between 15% to 32% relative to control. A phosphatase screen was performed. Out of 22 phosphatases screened, only three phosphatases showed inhibition of 35% to 40%.

Additional phosphatase inhibition profiles were obtained (presented in Table 4).

TABLE 4
Compound IC₅₀ Values (mean ± SE, μM)

| | IC₅₀ | | |
|---|---|---|---|
| Compound | Determination | DUSP5 (PD)-WT | PTPN1 |
| RR527 | Initial rates | >1,000 (using | Does not inhibit |
| (CSD3-2320) | | ERK2 as substrate) | |
| NCI2602 | Initial rates | >1,000 | >1,000 |
| (RR535) | | | |
| RR601 | Initial rates | 30.0 ± 3.0 | 2.5 ± 0.1 |
| (NCI2602 | | | |
| dimer) | | | |
| RR533 | Initial rates | 26.4 ± 7.2 | 106.0 ± 0.1 |

Other embodiments and uses of the invention will be apparent to those skilled in the art from consideration from the specification and the practice of the invention disclosed in this application. All references cited herein for any reason, including all journal citations and U.S./foreign patents and patent applications, are specifically and entirely incorporated herein by reference. It is understood that the invention is not confined to the specific reagents, formulations, reaction conditions, etc., herein illustrated and described, but embraces such modified forms as come within the scope of the following claims.

1. A pharmaceutical composition comprising a compound, or a pharmaceutically acceptable salt, solvate, or hydrate thereof, and a pharmaceutically acceptable carrier, wherein the compound comprises two negatively charged sulfonate groups or bioisosteric sulfonate analog groups tethered by a core scaffold of biphenyl or a planar analog of biphenyl, and wherein the core scaffold separates the negatively charged groups at a distance of about 6 to about 8 Angstroms.

2. The composition of claim 1, wherein the core scaffold comprises one or more of fluorene, biphenyl, carbazole, dibenzofuran, calixarene, and naphthalene.

3. The composition of claim 1, wherein the compound has a structure selected from the group consisting of

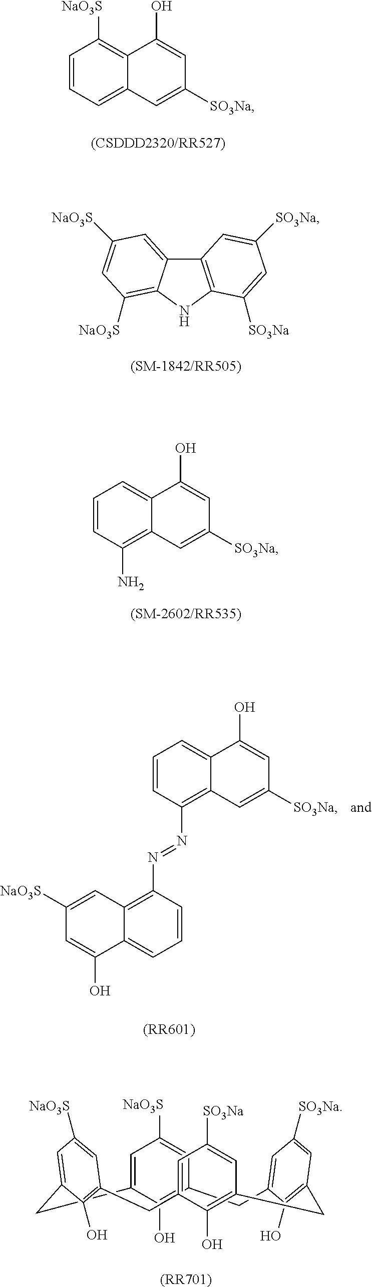

4. The composition of claim 1, wherein the negatively charged sulfonate groups or bioisosteric sulfonate analog groups are separated by a distance of about 7 angstroms.

5. The composition of claim 1, wherein the bioisosteric sulfonate analog groups are selected from the group consisting of sulfonamide, tetrazole, and carboxylic acid.

6. The composition according to claim 1, wherein said compound is a dual specificity phosphatase-5 (DUSP5) inhibitor.

7. A medicament for treating, preventing, or alleviating a vascular anomaly comprising at least one compound as defined in claim 1 in an effective amount to antagonize DUSP5.

8. A method of treating, preventing, or alleviating a vascular anomaly comprising administering to a subject in need thereof a therapeutically effective amount of a compound or composition defined in claim 1 wherein the compound antagonizes dual specificity phosphatase-5 (DUSP5).

9. The method of claim 7, wherein the vascular anomaly is associated with a Serine to Proline mutation at amino acid residue position 147 of DUSP5.

10. A method of screening a compound for effectiveness as an antagonist of DUSP5, the method comprising: a) exposing a sample comprising a DUSP5 polypeptide to a compound, and b) determining if DUSP5 phosphatase activity in said sample is decreased in comparison to a control sample lacking the compound.

11. Use of a DUSP5 inhibitor for treating, preventing, or alleviating vascular anomalies.

12. Use of a DUSP5 inhibitor for the preparation of a medicament for treating, preventing, or alleviating vascular anomalies.
